SEQUENCE LISTING (2 sequences)

SEQ ID NO: 1 - DNA, 23 bp, Artificial Sequence
  Description of Artificial Sequence; Note = Synthetic Construct
attgttcattgcctggggagttt

SEQ ID NO: 2 - DNA, 25 bp, Artificial Sequence
  Description of Artificial Sequence; Note = Synthetic Construct
atctggtgctcctcagatcagctaa

(19) United States
(12) Patent Application Publication
(10) Pub. No.: US 2002/0127208 A1
(43) Pub. Date: Sep. 12, 2002

(54) Method of transplantation using chemotherapy-treated allogeneic cells that enhance immune responses without graft versus host disease

(76) Inventors: Edmund K. Waller, Atlanta, GA (US); Christopher D. Hillyer, Dunwoody, GA (US); John Roback, Decatur, GA (US)

Correspondence Address: NEEDLE & ROSENBERG, P.C., The Candler Building, Suite 1200, 127 Peachtree Street, N.E., Atlanta, GA, 30303-1811, US

(21) Appl. No.: 09/945,339

(22) Filed: Aug. 31, 2001

Related U.S. Application Data
(60) Provisional application No. 60/229,593, filed on Aug. 31, 2000.

(51) Int. Cl. A61K 48/00; A61K 31/522; A61K 31/7115

(52) U.S. Cl. 424/93.21; 424/93.7; 514/45; 514/46; 514/263.3; 514/263.37; 514/50

(57) ABSTRACT

The present invention provides a method of transplanting hematopoietic cells between genetically unrelated individuals, comprising administering to the recipient, in combination with the administration of the hematopoietic cells, an amount of mononuclear cells which are treated so as to substantially reduce their ability to cause graft versus host disease while they retain their ability to proliferate in the recipient. The treated mononuclear cells can facilitate engraftment of hematopoietic cells when transplanted in combination with hematopoietic cells, treat or prevent infections, and treat cancer.

This application claims priority to U.S. provisional application Serial No. 60/229,593, filed Aug. 31, 2000, herein incorporated by reference in its entirety.

### BACKGROUND OF THE INVENTION

1. Field of the Invention

The present invention relates to a method of transplanting hematopoietic cells between genetically unrelated individuals using mononuclear cells treated so as to substantially reduce their ability to cause graft versus host disease while they retain their ability to proliferate in the recipient and facilitate engraftment by hematopoietic cells, treat or prevent infections, and treat cancer.

2. Background Art

Allogeneic bone marrow transplantation is the preferred treatment for a variety of malignant and genetic diseases of the blood and blood-forming cells. The widespread application of this therapy is limited by the availability of suitable bone marrow donors who are genetically related to the patient and share the same transplantation antigens on the surface of their blood cells. Only 25% of patients have a sibling who is an antigenically matched potential donor. Bone marrow ATTORNEY DOCKET NO. 05010.0087U1 transplantation can be offered to those patients who lack an appropriate sibling donor by using bone marrow from antigenically matched, genetically unrelated donors (identified through a national registry), or by using bone marrow from a genetically related sibling or parent whose transplantation antigens differ by one to three of six human leukocyte antigens from those of the patient. However, using antigenically mismatched, genetically related parent or sibling or antigenically matched, genetically unrelated donors, the likelihood of fatal graft versus host disease (GvHD) and/or graft rejection increases from 20% for matched sibling donors to 50% in the cases of matched, unrelated donors and unmatched donors from the patient's family. Further, in cases where an unrelated donor is not matched at one of the six major transplantation antigens, graft rejection and/or fatal GvHD increases to 60%.

GvHD is a disease with significant morbidity. Patients who develop acute GvHD may develop blisters covering most of their skin surface, massive gastrointestinal bleeding or fulminant liver failure and jaundice. Patients who develop chronic GvHD may develop scleroderma that results in joint contractures and skin ulcers, hair loss and a generalized wasting syndrome. Patients with acute or chronic GvHD are immuno-suppressed and at risk for life-threatening opportunistic infections similar to those that develop among AIDS patients.

The removal of T cells from the bone marrow obtained from matched unrelated or unmatched sibling donors results in a decreased incidence of graft vs. host reactions, but an increased incidence of rejection of the allogeneic bone marrow graft by the patient. Thus, lymphocytes, and especially T cells, present in the allogeneic bone marrow graft are important to ensure engraftment in antigenically and genetically mis-matched recipients. T cells present in the allogeneic graft also have an important role in eliminating residual cancer cells in the recipient, a phenomenon termed “graft versus leukemia effect.” The “ideal” donor T cell in an allogeneic bone marrow or stem cell graft would have the ability to prevent graft rejection and mediate the graft versus leukemia effect without producing GvHD. The potential to successfully transplant T cell-depleted, or stem cell-enriched bone marrow or stem cells from antigenically mis-matched donors to patients without graft rejection or GvHD would greatly extend the availability of bone marrow transplantation or solid organ transplantation to those patients without an antigenically matched sibling donor. A “solid organ transplant” means the transfer of a functioning organ from one individual to another.

In a dog model of allogeneic bone marrow transplantation, the addition of viable donor lymphocytes to the bone marrow graft resulted in an increased frequency of stable engraftment, from 9% using antigenically mismatched bone marrow alone, to 88%, using a combination of bone marrow and donor lymphocytes. However, all the animals that received donor lymphocytes died of lethal GvHD (Storb et al. (1968) “Marrow grafts by combined marrow and leucocyte infusions in unrelated dogs selected by histocompatability typing” Transplantation 6:587-593).

An alternative to infusions of viable donor lymphocytes has been the use of irradiated donor lymphocytic infusions in the post-transplant period. The addition of donor lymphocytes that had been previously irradiated to a dose of 20 cGy (2,000 rads) to allogeneic bone marrow cells did not prevent fatal graft failure when the mixture was administered to lethally irradiated dogs antigenically mismatched for dog leukocyte antigens (DLA) (Deeg et al. (1979) “Abrogation of resistance to and enhancement of DLA-nonidentical unrelated marrow grafts in lethally irradiated dogs by thoracic duct lymphocytes” Blood 53:552-587).

In genetically unrelated rabbits, a series of five infusions of donor lymphocytes, irradiated to 15 cG (1,500 rads) one to ten days following the infusion of allogeneic bone marrow cells and irradiated autologous bone marrow cells decreased the rate of graft rejection from 60% to 20%, but only 30% of the treated animals survived more than 100 days with donor derived hematopoietic cells and 40% of animals that received T cell depleted bone marrow followed by irradiated allogeneic lymphocytes developed GvHD. (Gratwohl et al. (1987) “Engraftment of T-cell depleted rabbit bone marrow” Acta haematol. 77:208-214).

The addition of antigenically matched viable donor lymphocytes obtained from the bone marrow donor and given 1-5 days post-transplant to 43 patients undergoing allogeneic bone marrow transplantation for aplastic anemia resulted in a 14% incidence of graft failure compared to 22% in a similar group of 20 patients who received the bone marrow transplant without additional donor lymphocytes, to 14% in the group that received post-transplant donor lymphocytes. However, the incidence of acute GvHD was 36% in the group treated with donor lymphocytes compared to a 20% incidence of acute GvHD in the group that received bone marrow cells alone. In both groups, 20% of patients ultimately died of GvHD (Storb et al. (1982) “Marrow transplantation with or without donor buffy coat cells for 65 transfused aplastic anemia patients” Blood 59:236-246).

In a clinical report describing the use of irradiated human lymphocytes, 20 patients with hematological malignancies were treated with high-dose chemotherapy and total body irradiation followed by the infusion of T cell-depleted antigenically matched, genetically related allogeneic bone marrow cells. One, three, five, seven and fourteen days following bone marrow transplantation, the patients received infusions of donor lymphocytes, irradiated to 15 cGy (1,500 rads). The authors reported no cases of graft failure, but noted an overall incidence of GvHD of 85% and a 15% incidence of fatal GvHD (Gratwohl et al (1988) “Irradiated donor buffy coat following T cell-depleted bone marrow transplants” Bone Marrow Transplantation 3:577-582).

It has been demonstrated that irradiated donor T-cells have the ability to facilitate engraftment of allogeneic BM cells without causing GvHD in the recipients¹. This approach has been applied in clinical trials with promising results 2 but may have limited clinical application because infusions of large numbers of irradiated splenocytes result in persistent mixed chimerism in a murine model.¹ Recipients of allogeneic transplant with persistent mixed chimerism in the T-cell compartment have less of the GvL effect than transplanted recipients that achieve full donor chimerism³.

A successful method for the ex vivo treatment of donor T-cells to limit their ability to cause graft-versus-host disease (GvHD) while preserving graft-versus-leukemia effects (GvL) would have broad clinical application for patients undergoing allogeneic hematopoietic cell transplantation for malignant or benign disease. The ideal graft would include sufficient donor hematopoietic progenitor cells for rapid hematopoietic engraftment, and donor T-cells that can contribute to post-transplant immune reconstitution and GvL effects without causing GvHD.

Fludarabine (9-beta-D-arabinosyl-2-fluoroadenine) is an adenine nucleoside analog which inhibits DNA synthesis when incorporated into a replicating chain^{4,5}. Fludarabine inhibits proliferative responses of human CD4+ and CD8+ T-cells to mitogen and allo-antigen⁶, and has potent immunosuppressive effects including profound depletion of CD4+ T-cells 7 and induction of apoptosis in resting lymphocytes . Fludarabine also inhibits STAT 1 signaling in resting T-cells, leading to decreased proliferation in response to cytokine activation⁹. Thus, the immunosuppressive effects of fludarabine can decrease the responsiveness of donor T-cells to allogeneic stimulation in a transplant setting, yet preserve their capacity to contribute to long-term donor derived hematopoiesis.

### SUMMARY OF THE INVENTION

The present invention provides a method of transplanting hematopoietic cells from a donor source into a genetically unrelated recipient, comprising administering to the recipient, in combination with the administration of the hematopoietic cells, an amount of mononuclear cells which are treated so as to substantially reduce their ability to cause graft versus host disease while they retain their ability to proliferate in the recipient and facilitate engraftment of the hematopoietic cells in the recipient; and administering to the recipient an effective amount of hematopoietic cells.

The present invention provides a method of treating or preventing an infection in a recipient of genetically unrelated hematopoietic cells, comprising administering to the recipient, in combination with the administration of the hematopoietic cells, an amount of mononuclear cells which are treated so as to substantially reduce their ability to cause graft versus host disease while they retain their ability to proliferate in the recipient, and which are effective in treating or preventing the infection.

Also provided by the present invention is a method of enhancing immune reconstitution in a transplant recipient, comprising administering to the recipient, in combination with a transplant, an amount of mononuclear cells which are treated so as to substantially reduce their ability to cause graft versus host disease while they retain their ability to proliferate in the recipient, and which are effective in enhancing immune reconstitution in the recipient.

The present invention also provides a method of enhancing immune reconstitution in a subject diagnosed with cancer, comprising administering to the subject an amount of mononuclear cells which are treated so as to substantially reduce their ability to cause graft versus host disease while they retain their ability to proliferate in the subject, and which are effective in enhancing immune reconstitution in the subject.

The present invention provides a method of treating or preventing an infection in a genetically unrelated solid organ transplant recipient, comprising administering to the recipient, in combination with the transplant, an amount of mononuclear cells which are treated so as to substantially reduce their ability to cause graft versus host disease while they retain their ability to proliferate in the recipient, and which are effective in treating or preventing the infection.

Also provided by the present invention is a method of treating or preventing an infection in a subject, comprising administering to the subject an amount of mononuclear cells which are treated so as to substantially reduce their ability to cause graft versus host disease while they retain their ability to proliferate in the subject, and which are effective in treating or preventing the infection.

The present invention also provides a method of treating cancer in a subject diagnosed with a cancer, comprising administering to the subject an amount of mononuclear cells which are treated so as to substantially reduce their ability to cause graft versus host disease while they retain their ability to proliferate in the subject, and which are effective in treating the cancer.

### BRIEF DESCRIPTION OF THE DRAWINGS

FIGS. 1A-B. Fludarabine treatment attenuates the GvHD potential of allogeneic donor lymphocytes. Lethally irradiated (11 Gy) B10.BR (H2^{k}) recipient mice were transplanted with 1×10⁶ BL6 (H2^{b}) TCD-BM cells (n=40, thin line). Additional groups received BL6 TCD BM supplemented with 1×10⁷ BL6 splenocytes recovered from 24 hours culture in complete media (n=20, thick dashes) or media containing 20 μg/ml fludarabine (n=35, thick line), or TCD BM plus 6×10⁷ irradiated B6 splenocytes (n=15, thin dashes). Survival and weight change data are derived from 2-6 individual experiments using 5 or 10 animals per group. Kaplan-Meier survival determinations are shown in A (ticks indicate time points at which individual experiments were terminated). Trend lines representing the mean weight change (percent body weight) for each group are shown in B. Trend lines were calculated using a moving average with a periodicity of 3.

FIGS. 2A-B. Fludarabine-treated donor spleen cells facilitate rapid and durable donor engraftment and lead to more complete donor chimerism than irradiated lymphocytes.

A. Percent donor chimerism in peripheral blood leukocytes.

B. T-cell hematopoiesis of surviving recipients at 1 month and 2 to 3 months post-BMT. Congenically marked donor lymphocytes were used to differentiate T-cells of donor BM (CD45.2) and donor SP (CD45.1) origin. X-axis labels identify groups which received allogeneic TCD-BM alone, or with addition of irradiated splenocytes or fludarabine treated splenocytes. Data are compiled from 3-4 separate experiments. Blood leukocyte chimerism was analyzed in a total of 10-23 mice for each group at the two time points. Error bars represent the standard error of the mean.

FIGS. 3A-C. Fludarabine treatment improves the engraftment/GvHD activity ratio of allogeneic donor splenocytes.

A. Engraftment of allogeneic splenocyte-derived T-cells increases with administered cell dose. 1×10⁶ TCD-BM cells and splenocytes from BL6 strains having different Thy1.1 or Thy1.2 isotypes were administered to lethally irradiated B10.BR mice. Varying numbers of fludarabine treated (20 μg/ml, 24 hr) or untreated splenocytes (0 μg/ml, 24 hr) were administered as indicated, i.e., 1 Flu.=1×10⁶ fludarabine treated splenocytes, 3 Unt.=3×10⁶ untreated splenocytes. 20 mice were transplanted per group, except as noted. Donor splenocyte derived CD4+ (gray bars) and CD8+ (black bars) were quantified in the peripheral blood of recipient mice 2-4 months post-transplant. The number of mice analyzed for each group was equal to the day 60 survival as follows: 1 Flu, n=18; 3 Flu, n=19; 10 Flu, n=13; 1 Unt, n=19; 3 Unt, n=17. Only 10 mice were transplanted in the 8 Unt. group; day 60 survival was 4/10, and 2 mice were analyzed for T-cell engraftment. Data are not shown for mice which received TCD-BM alone (day 60 survival=3/5).

B. Fludarabine treated donor T-cells have one half the engraftment potential of untreated T-cells. The number of donor splenocyte-derived CD8+ T-cells from FIG. 3A is plotted against the number of untreated (∘) or fludarabine treated splenocytes (●) administered. Trend lines were fit to the data with the y intersect set to zero, and resulting equations are shown.

C. Fludarabine treated donor T-cells have one third the GvHD potential compared to untreated T-cells. The average weight of surviving mice at day 60 post-transplant is plotted against the number of untreated (∘) or fludarabine treated splenocytes (●) administered. Trend lines were fit to the data, and the resulting equations are shown.⁵⁵⁴ The mean weight loss at day 40 was used for this group (7/10 mice surviving) in order to provide a better representation of GvHD. Day 50-60 survival was 4/10. Error bars represent the standard error of the mean.

FIGS. 4A-C. Fludarabine treatment reduces alloreactivity and cytotoxicity of allogeneic splenocytes in vitro.

A. Fludarabine attenuates the proliferative response of spleen cells to allogeneic stimulation (MLR) or mitogenic stimulation (con A). Effect of fludarabine on proliferation of responder splenocytes in a 7-day MLR containing 20,000 responder cells and 10,000 irradiated (25 Gy) allogeneic stimulator cells (n=4; p<0.05), or 20,000 responder cells incubated in the presence of 10 μg/ml concanavalin A (n=3; p<0.05). Strain combinations of DBA versus BL6 and BL6 versus DBA were used in the MLR experiments. CPM is expressed as percent of non-treated control.

B. Fludarabine reduces the frequency of alloreactive T-cells. Limiting dilution assay of B10.BR responder cells stimulated with C57.BL6. The frequencies of allo-reactive cells in untreated and fludarabine treated cultures are shown. Other strains were also tested yielding similar reduction in alloreactive T-cell frequency with 20 μg/ml fludarabine (percent reduction values ranging from 25% to 67%; n=5).

C. Fludarabine exposure prior to, but not after, allo-stimulation reduces the cytotoxic potential of allogeneic splenocytes. Data for an Effector (CTL) to Target (C1498) ratio of 20:1 are expressed as percent of non-treated control. Cytotoxicity of spleen cells treated with 20 μg/ml fludarabine either 1 day before stimulation with allo-antigen (n=S, p<0.05) or 10 days after allo-priming (n=2, not significant) in a 10-day MLR. Data are compiled from experiments using DBA (H2^{d}), Balb/C (H2^{d}) or B10.BR (H2^{k}) splenocytes as effector cells and C57BL6 (H2^{b}) splenocytes as target cells. Error bars represent the standard error of the mean.

FIG. 5. T-cells from engrafted recipients are tolerant to both recipient- and donor-type allo-antigens but retain reactivity to third party type antigen and mitogen. Proliferative response of spleen cells harvested from B10.BR mice 8 months after transplant with 1) C57BL/6 TCD BM only, 2) C57BL/6 TCD BM+fludarabine-treated splenocytes, and 3) C57BL/6 TCD-BM+B10.BR splenocytes. Proliferation is measured after stimulation with irradiated splenocytes from donor-type (C57BL/6, H2^{b}), recipient-type (B10.BR, H2^{k}), and third party (DBA/2, H2^{d}) mice, and concanavalin A. 400,000 responding cells were cultured with 200,000 irradiated stimulators in a 7-day MLR.

FIG. 6. Graft facilitation is mediated by T-cells. B10.BR recipients were lethally irradiated and transplanted with 1×10⁶ TCD-BM from CD45.1+ C57.BL6 donors. Different groups received 1×10⁷ splenocytes from congenic CD45.2+C57.BL6 (WT) mice, NK deficient beige (BG) mutants, or T-cell depleted C57.BL6 (TCD) splenocytes, after 24hr untreated (Untr.) or fludarabine treated (Flu.) culture. The fraction of blood T-cells derived from recipient, donor BM, and donor spleen were determined at day +27 post-transplant, using markers for T-cells (CD3), recipient-type cells (H2^{k}), donor-type cells (H2^{b}), and CD45.1/CD45.2 isoforms.

FIGS. 7A-B. Fludarabine treated allogeneic donor lymphocytes retain significant GvL activity.

A. Survival ofC57.BL6 mice co-transplanted with B10.BR TCD-BM and C1498 myeloid leukemia cells. Each group of mice received either: no added splenocytes (n=5, thin line), untreated splenocytes (n=5, thick dashes), irradiated splenocytes (n=10, thin dashes), or fludarabine-treated splenocytes (n=10, thick line), on the day of transplant.

B. Survival of B10.BR mice co-transplanted with C57.BL6 TCD-BMand LBRM lymphoma cells. Each group of 10 mice received either: no added splenocytes (thin line), or weekly infusions of 1×10⁷ untreated splenocytes (thick dashes), irradiated splenocytes (thin dashes), or fludarabine-treated splenocytes (thick line) days 0, 7, 14, 21 and 28. The results shown are obtained from a single experiment. Two additional experiments using either the same strain combinations or C57.BL6→(C57.BL6×B10.BR F1 hybrid) showed similar results, i.e., groups which received fludarabine treated splenocytes had a survival advantage.

FIGS. 8A-B. MCMV lethality following BMT. 5×10⁶ TCD BM cells from either syngeneic F1 (B/6× BALB/c) (A) or allogeneic B/6 mice (B) were infused into conditioned F1 recipients, followed by infection with MCMV at inoculae of 10⁴ (open circle), 5×10⁴ (closed triangle), 10⁵ (open triangle), 5×10⁵ (closed box), 10⁶ (open box), or 5×10⁶ (closed diamond) PFU (n=10 mice per inoculum). Mice were evaluated daily to score lethality. All BMT recipients infused with vehicle alone (closed circle) survived. Mortality of allogeneic BMT recipients was significantly higher than syngeneic recipients following infection with MCMV at doses of 10⁴ (P<0.001), 5×10⁴ (P<0.001), 10⁵ (P<0.005), and 5×10⁵ PFU (P<0.025) by the Mantel-Cox log-rank test. Data from two separate experiments were combined for display and analysis.

FIGS. 9A-C. Adoptive immunotherapy to prevent MCMV lethality. Conditioned F1 recipients were infused with 5×10⁶ TCD BM cells from B/6 donors. Some groups also received simultaneous infusions of 30×10⁶ unselected splenocytes from B/6 donors previously infected with MCMV (immune). Prior to infusion, the splenocytes were either maintained in vitro in culture media for 24 hours as an untreated control (open triangle), or treated with γ-irradiation (closed triangle), S-59/UVA (closed circle), or fludarabine (open circle). Control mice did not receive splenocyte infusions (closed square). Following transplantation, mice were either infected with 2×10⁴ PFU MCMV (A), or vehicle (B). Data from two separate experiments (n=20 mice per group) were combined for display and analysis. Compared to MCMV-infected BMT mice (A) that did not receive splenocyte infusions, infusion of psoralen-treated, γ-irradiated, or fludarabine-treated immune splenocytes significantly improved survival (P<0.001 by Mantel-Cox log-rank test). Survival was not improved when mice were infused with cultured (untreated) splenocytes (P>0.05). The difference in survival between MCMV-infected mice receiving splenocytes treated with either psoralen, fludarabine, or y-irradiation was not significant (P >0.05). In the absence of MCMV infection (B), BMT mice receiving cultured splenocytes had significantly greater mortality that control mice that did not receive splenocytes (P<0.001). Infusion of treated splenocytes did not significantly affect survival in the absence of MCMV infection (P>0.05). In uninfected BMT mice, the survival was significantly greater (P<0.01) in mice receiving psoralen-treated or irradiated splenocytes as compared to those receiving fludarabine-treated splenocytes.

The mean percentage change in weight of surviving recipients of allogeneic transplants [C57.BL6→F1(C57.BL6×Balb/c)] was measured twice weekly. Treatment groups received untreated, irradiated, fludarabine-treated, or psoralen-treated donor splenocytes in combination with TCD bone marrow and a post-transplant inoculum of MCMV. Twice weekly measurements of mean weights post-transplant demonstrated substantial and progressive weight loss, consistent with the development of graft versus host disease, among recipients of cultured, non-treated splenocytes (C). Recipients of psoralen-treated and irradiated splenocytes had no significant increased weight loss compared to mice that received no additional splenocytes. Recipients of fludarabine-treated splenocytes had a degree of weight loss intermediate between recipients of no splenocytes and recipients of untreated splenocytes (C).

FIG. 10. Adoptive immunotherapy decreases hepatic MCMV load. B/6→F1 BMT mice received adoptive transfer of 30×10⁶ MCMV-immune lymphocytes followed by infection with 5×10⁴ PFU MCMV. Surviving mice were sacrificed at day +14, and segments of liver were subject to PCR quantitation of MCMV load. Adoptive immunotherapy significantly reduced the average hepatic viral load by 4-5 log₁₀ (P<0.05), depending on the method used for lymphocyte pretreatment. The difference in viral load between mice protected with S-59/UVA and fludarabine-treated lymphocytes was also significant (P<0.005).

FIGS. 11A-H. S-59/UVA-treated lymphocytes decrease hepatic MCMV infection without producing GvHD. Allogeneic BMT recipients were divided into groups that either did not receive adoptive immunotherapy (A, B, G), or received passive transfer of 30×10⁶ MCMV-immune lymphocytes that were untreated (C, D) or pretreated with S-59/UVA (E, F). Some mice were infected with 5×10⁴ PFU MCMV (B, D, F, G), while others were uninfected (A, C, E). At day +14, livers were harvested from surviving mice and prepared for histologic examination. Representative sections are displayed. Mice that did not receive adoptive immunotherapy displayed histologically normal-appearing liver in the absence of MCMV infection (A), but showed markedly distorted architecture, necrosis, and abundant inflammation following infection (B). Numerous typical cytomegalic inclusions can be appreciated in a higher power photomicrograph (G). MCMV inclusions were only identified in MCMV infected mice that did not receive adoptive immunotherapy, with an average of 46 viral inclusions per 10 high-power fields (H). Adoptive transfer of untreated lymphocytes p [rotected against MCMV viral effect (D,H), but these mice showed marked hepatic inflammation both in the absence (C) or presence of MCMV infection (D). In contrast, mice infused with S-59/UVA lymphocytes had normal-appearing hepatic architecture with minimal detectable inflammation whether they were infected (F) or not (E). Importantly, these mice showed significantly less hepatic inflammation than mice receiving untreated lymphocytes (H).

FIG. 12. S-59/UVA treated T-cells persist long-term following adoptive transfer. Flow cytometry was used to quantitate the contribution of the BMT recipient, BMT donor, and adoptive immunotherapy donor to the peripheral T-cell compartment at day +90. Approximately 50% or more of the peripheral T-cells were derived from the BM donor in all groups. However, the contribution of the BMT recipient and the lymphocyte donor varied, based on the lymphocyte pretreatment regimen and concurrent MCMV infection. Adoptively transferred fludarabine-treated lymphocytes accounted for approximately 50% of peripheral T-cells, and no host-derived T-cells were detected. In contrast, gamma-irradiated lymphocytes accounted for less than 15% of the total, and the mice were not converted to full donor chimerism. S-59/UVA treated T-cells made up 35% of the total in the presence of MCMV infection, but only 8% in the absence of infection, suggesting that MCMV infection may increase persistence of adoptively transferred T-cells despite photochemical treatment. The rare T-cells identified as being of lymphocyte donor origin in the mice that did not receive adoptive immunotherapy are within the experimental variation of the assay.

### DETAILED DESCRIPTION OF THE INVENTION

It must be noted that, as used in the specification and the appended claims, the singular forms “a,” “an,” and “the” include plural referents unless the context clearly dictates otherwise. Thus, for example, reference to “a chemotherapeutic agent” can also include more than one particular species of chemotherapeutic agent.

The present invention provides a method of transplanting hematopoietic cells from a donor source into a genetically unrelated recipient, comprising administering to the recipient, in combination with the administration of the hematopoietic cells, an amount of mononuclear cells which are treated so as to substantially reduce their ability to cause graft versus host disease while they retain their ability to proliferate in the recipient and facilitate engraftment of the hematopoietic cells in the recipient; and administering to the recipient an effective amount of hematopoietic cells. By “in combination” is meant the treated mononuclear cells can be administered to the recipient before, contemporaneously with, or after the administration of the hematopoietic cells to the recipient. As used herein, the phrase “substantially reduce their ability to cause graft versus host disease” means that as a result of treatment according to the methods of the invention, a given concentration of treated mononuclear cells administered to a recipient results in the absence of graft versus host disease, whereas the same concentration of untreated mononuclear cells administered to a recipient results in severe graft versus host disease.

The present invention also provides a method of transplanting hematopoietic cells from a donor source into a genetically unrelated recipient, comprising administering to the recipient, in combination with the administration of the hematopoietic cells, an amount of mononuclear cells which are treated so as to render them incapable of causing graft versus host disease but not incapable of proliferation, and which are effective in enhancing engraftment of the hematopoietic cells in the recipient; and administering to the recipient an effective amount of hematopoietic cells. By “proliferation” is meant cell division that leads to an increase in the number of nucleated cells such that the number of cells and their progeny at, for example, time T+1 is greater than the number of cells at time T.

As used herein, “hematopoietic cells” means a population of cells, preferably human, that possesses the capability of dividing and producing progeny that include all of the formed cellular elements of the blood. As used herein, “donor” means the animal, preferably human, that is the natural source from which the hematopoietic cells are originally removed. Also as used herein, a “recipient” is the animal, typically human, into which the hematopoietic cells will be transplanted. The term “allogeneic” as used herein means that the recipient is not the natural source from which the hematopoietic cells have been removed. Major histocompatability complex antigens (also called human leukocyte antigens, HLA) are protein molecules expressed on the surface of cells that confer a unique antigenic identity to these cells. MHC/HLA antigens are target molecules that are recognized by certain immune effector cells (T-cells and natural killer (NK) cells) as being derived from the same source of hematopoietic reconstituting stem cells as the immune effector cells (“self”) or as being derived from another source of hematopoietic reconstituting cells (“non-self”).

Mononuclear cells are cells of the hematopoietic system identified by a round, nonsegmented nucleus. Mononuclear cells can include T cells, NK cells, monocytes, mixtures of T cells, NK cells or monocytes.

Properties of T cells include the expression of a complex of proteins on their cell surfaces that include the CD3 antigen and the T cell receptor (TCR) that can bind to MHC/HLA molecules expressed on the surface of other cells. The presence of the CD3/TCR complex allows T cells to recognize cells from genetically different individuals as expressing “non-self” MHC/HLA antigens and to recognize virally infected cells and tumor cells from the same individual as expressing “altered self” MHC/HLA antigens. T cells are able to bind to and kill cells that express “non-self” and “altered self” MHC/HLA by the activation of specific cytolytic enzymes; they regulate (including stimulation and inhibition) T cell and B cell proliferation and antibody production in response to a specific antigen; they release protein molecules called cytokines that stimulate or inhibit the immune response; and they undergo multiple rounds of cell division and produce daughter cells with similar biologic properties as the parent cell. Examples of T cells with some attributes of NK cells include cells that express both the CD3 (T cell specific) and the CD56 (NK cell specific) antigens.

Properties of NK cells include the expression of antigens on their cell surface that include one or more of the following: CD16, CD56, and CD57 and the absence of the alpha/beta or gamma/delta TCR complex expressed on the cell surface; the ability to bind to and kill cells that fail to express “self” MHC/HLA antigens by the activation of specific cytolytic enzymes; the ability to kill tumor cells from a genetically unrelated individual; the ability to release protein molecules called cytokines that stimulate or inhibit the immune response; and the ability to undergo multiple rounds of cell division and produce daughter cells with similar biologic properties as the parent cell. Properties of monocytes include the ability to engulf bacteria and “non-self” cells (phagocytosis); the elaboration of cytokines that stimulate T cells and NK cells; the release of molecules that cause inflammation; and the presentation of antigens to T cells.

The mononuclear cells can be treated with a chemotherapeutic agent, for example a nucleoside analog or a psoralen activated by ultraviolet A (UVA) light, in an amount that is sufficient to attenuate the potential of the mononuclear cells to produce graft versus host disease (GvHD) but not sufficient to render the cells incapable of proliferation in the recipient. Thus, the mononuclear cells can be treated so as to substantially reduce their ability to cause graft versus host disease while they retain their ability to proliferate in the recipient. A nucleoside analog is an analog of a purine or pyrimidine that is incorporated into newly synthesized RNA or DNA by a polymerase enzyme. As used herein, the phrase “attenuate the potential of the mononuclear cells to produce graft versus host disease” means reducing the ability of donor mononuclear cells to cause GvHD; reducing the incidence and/or severity of GvHD in a population of allogeneic transplant recipients.

In another embodiment of the invention, the mononuclear cells can be treated by exposure to cytotoxic chemotherapeutic agents to render the cells incapable of causing GvHD, but capable of proliferating in a host to augment the immune system of the host. Cytotoxic chemotherapeutic drugs act by cross-linking DNA (e.g., psoralens activated by UVA light) or otherwise interfering with normal cellular metabolism (nucleoside analogs) so as to inhibit the proliferative capacity of cells (Chabner (1993) “Anticancer Drugs” in Cancer: Principles and Practice of Oncology, Fourth Edition, eds. DeVita, Hellman, and Rosenberg. pp. 325-417. J. B. Lippincott publishers, Philadelphia). The phrase “render cells incapable of proliferation” means to reduce by at least 90% the cell division that leads to an increase in the number of nucleated cells such that the number of cells and their progeny at time T+1 is greater than the number of cells at time T. A concentration of the drug can be used that allows the cells to retain the capacity for proliferation while having reduced capacity for producing graft versus host disease effects. Examples of such cytotoxic chemotherapeutic drugs that could be employed in the present invention include, but are not limited to, mitomycin C, bleomycin, actinomycin D, doxirubicin, daunorubicin, mitoxanthrone, cytarabine, streptozocin and amsacrine, 9-D-arabinofuranosyl-2-fluoroadenosinemonophosphate (fludarabine), 2′-deoxcoformycin (pentostatin), 2-chlorodeoxyadenosine (2CDA), 6-mercaptopurine (6-MP), 6-thioguanine (6-TG), 2′-deoxy-2′,2′-difluorocytidine (gemcitabine) and 2-amino-9-D-arabinosyl-6-methoxy-9-H-purine (Ara-G, 506U78) and a psoralen, for example, S-59 psoralen activated by UVA light. The mononuclear cells are incubated with a sufficient concentration of the cytotoxic drug so as to substantially reduce their ability to cause graft versus host disease while they retain their ability to proliferate in the recipient to enhance the recipient's ability to fight cancer, leukemia and viral infection. A “sufficient concentration” is that which causes greater than 90% inhibition of the proliferation of treated cells as measured by assays known in the art.

The present invention also provides a method of treating or preventing an infection in a recipient of genetically unrelated hematopoietic cells, comprising administering to the recipient, in combination with the administration of the hematopoietic cells, an amount of mononuclear cells which are treated so as to substantially reduce their ability to cause graft versus host disease while they retain their ability to proliferate in the recipient, and which are effective in treating or preventing the infection. The infection can be caused by a bacterium, fungus, or a virus. For example, the virus can be cytomegalovirus.

The present invention also provides a method of preserving long-term immunological activity and proliferative capacity of allogeneic mononuclear cells and limiting their ability to produce graft versus host disease in a subject receiving a transplant, comprising contacting the cells with an effective dose of a chemotherapeutic agent, for example, a nucleoside analog ex vivo. By “proliferative capacity” is meant the ability of allogeneic mononuclear cells to proliferate (undergo proliferation). As used herein, “long-term immunological activity” means the presence of antigenic specific T-cells and B-cells that respond to antigen with activation and/or proliferation. Examples of immunological activity include, but are not limited to, cytotoxicity and synthesis of cytokines or chemokines. As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, a human.

Also provided by the present invention is a method of enhancing immune reconstitution in a transplant recipient, comprising administering to the recipient, in combination with a transplant, an amount of mononuclear cells which are treated so as to substantially reduce their ability to cause graft versus host disease while they retain their ability to proliferate in the recipient, and which are effective in enhancing immune reconstitution in the recipient. As used herein, “immune reconstitution” means the presence of long-term immunological activity following a period of suppressed immune function. By “in combination” is meant the treated mononuclear cells can be administered to the recipient before, contemporaneously with, or after the recipient receives the transplant. By “transplant” is meant an allogeneic or xenogeneic organ or tissue that is not native to the recipient.

The present invention also provides a method of treating cancer in a subject diagnosed with a cancer, comprising administering to the subject an amount of mononuclear cells which are treated so as to substantially reduce their ability to cause graft versus host disease while they retain their ability to proliferate in the subject, and which are effective in treating the cancer. For example, the cancer can be leukemia.

The present invention also provides a method of enhancing immune reconstitution in a subject diagnosed with cancer, comprising administering to the subject an amount of mononuclear cells which are treated so as to substantially reduce their ability to cause graft versus host disease while they retain their ability to proliferate in the subject, and which are effective in enhancing immune reconstitution in the subject. By “enhancing immune reconstitution” is meant causing an increase in the numbers of activities of antigen-specific immune cells, specifically T-cells and B-cells, as determined by assays known in the art. Enhancement of the immune system can be used to treat the underlying cancer and/or infection which can occur in a subject with cancer. Examples of cancers that can be treated according to the methods of the present invention include sarcoma, skin, brain, esophageal, stomach, liver, kidney, colon, bladder, prostate, ovarian, uterine, testicular, neuroendocrine, bone, and pancreatic cancer. The method is especially useful for hematopoietic cell cancers such as leukemias, lymphomas and multiple myeloma. The cancer can be localized or metastatic. The step of administering hematopoietic cells is not necessary to treat a cancer.

For treating a cancer, an allogeneic transfer of mononuclear cells is utilized although the transfer can also be xenogeneic. In either context, the tumor antigen or whole cells containing the antigen can be utilized to prime the donor or cells from the donor prior to transfer to the recipient. Current Protocol in Immunology ed. J. E. Coligan et al., John Wiley and Sons (1994).

The present invention provides a method of treating or preventing an infection in a genetically unrelated solid organ transplant recipient, comprising administering to the recipient, in combination with the transplant, an amount of mononuclear cells which are treated so as to substantially reduce their ability to cause graft versus host disease while they retain their ability to proliferate in the recipient, and which are effective in treating or preventing the infection. By “solid organ” is meant any tissue or organ that is not bone marrow or blood cells. Examples of solid organ transplants include, but are not limited to, liver, bone, nerve, heart, pancreas, skin, lung, cartilage and kidney. The infection can be caused by a bacterium, fungus, or virus. For example, the virus can be cytomegalovirus.

The methods of the present invention can be used to treat an immunocompetent or immunocompromised subject who is not a transplant recipient. For example, an immunocompetent HIV-positive subject, an immunocompromised HIV-positive subject, a neonate, and a subject that requires augmentation of cellular immunity can be treated by the methods described herein. A subject who requires augmentation of cellular immunity can have congenital or inherited immunodeficiency. Therefore, the present invention provides a method of treating or preventing an infection in a subject, comprising administering to the subject an amount of mononuclear cells which are treated so as to substantially reduce their ability to cause graft versus host disease while they retain their ability to proliferate in the subject, and which are effective in treating or preventing the infection. Specifically, the infection can be caused by a virus, for example, cytomegalovirus.

According to the methods of the present invention, the treated mononuclear cells can condition the recipient to successfully accept the transplanted cells and without mounting an immune response against the recipient's cells. The mononuclear cells can also exert a graft versus leukemia effect by which they aid in the elimination of residual cancer cells in the recipient.

The treated mononuclear cells can be administered to the recipient contemporaneously with or after the administration of the hematopoietic cells. Alternatively, the treated mononuclear cells can be administered to the recipient at any time before the administration of the hematopoietic cells and, in one embodiment, are administered up to ten days prior to administration of the hematopoietic cells. In another embodiment, the treated mononuclear cells are administered to the recipient between one and five days prior to the administration of the hematopoietic cells. Any range of treatment, e.g., one to nine, two to eight, three to seven, one to two, one to three, zero to one, zero to two days, etc. is also provided.

The hematopoietic cells and the treated mononuclear cells can be from the same donor source or they can be from different donors. These donor source cells include cells which are propagated in vitro or derived in vitro from a less differentiated cell type of the donor source, for example, from a yolk sac or other embryonic fetal tissue source such as embryonic stem cells.

The amount of treated mononuclear cells administered to the recipient, in one example, can be between about 0.05×10⁶ and about 100×10⁶ cells/Kg of the recipient's body weight. Subranges of treated mononuclear cells/Kg of the recipient's body weight are also provided, for example between about 5×10⁶ and about 25×10⁶, between about 10×10⁶ and about 20×10^{6,} between about 5×10⁶ and about 20×10⁶, between about 20×10⁶ and about 100×10⁶, etc.

The hematopoietic cells administered to the recipient can, in one example, be present in a source population of between about 0.2×10⁸ and about 6.0×10⁸, or ranges there between, donor bone marrow cells/Kg of the recipient's body weight. The bone marrow cells can be obtained from the donor by standard bone marrow aspiration techniques known in the art. Bone marrow cells are removed from the donor by placing a hollow needle into the marrow space and withdrawing a quantity of marrow cells by aspiration.

Alternatively, the hematopoietic cells administered to the recipient can, in one example, be present in a source population of between about 1.0×10⁸ and about 40×1⁸, or ranges there between, donor cytokine mobilized peripheral blood stem cells/Kg of recipient's body weight. Peripheral blood cells can be obtained from the donor, for example, by standard phlebotomy or apheresis techniques. Phlebotomy is performed by placing a hollow needle into a vein and withdrawing a quantity of whole blood using aspiration or gravity. Apheresis is performed in a similar manner to phlebotomy except the whole blood is anticoagulated and then separated into the constituent formed cellular elements by centrifugation. The mononuclear cell fraction is retained, and the remaining plasma and other cellular elements (red blood cells, granulocytes, platelets) are returned to the patient by intravenous infusion.

Peripheral blood stem cells can be cytokine mobilized by injecting the donor with hematopoietic growth factors such as FLT-3, Granulocyte colony stimulating factor (G-CSF), granulocyte-monocyte colony stimulating factor (GM-CSF), and/or stem cell factor (SCF) subcutaneously or intravenously in amounts sufficient to cause movement of hematopoietic stem cells from the bone marrow space into the peripheral circulation. The hematopoietic reconstituting cells can also be derived from fetal or embryonic human tissue that is processed and/or cultured in vitro so as to increase the numbers or purity of primitive hematopoietic elements.

The hematopoietic cells administered to the recipient can be T cell-depleted to prevent the development of GvHD. The cell population is depleted of T cells by one of many methods known to one skilled in the art (Blazer et al., (1985) “Comparison of three techniques for the ex vivo elimination of T cells from human bone marrow.” Experimental Hematology 13:123-128) or by using affinity chromatography, as described below. In addition, the hematopoietic cells administered to the recipient can also be hematopoietic system cells that have been enriched from the source population. The source population can be either donor bone marrow cells or donor peripheral blood cells. The hematopoietic cells can be enriched from the source population by selecting cells that express the CD34 antigen, using combinations of density centrifugation, immuno-magnetic bead purification, affinity chromatography, and flourescent activated cell sorting (FACS), known to those skilled in the art (Baum, C. M., I. L. Weissman et al., (1992) “Isolation of a candidate human hematopoietic stem-cell population” Proc. Natl. Acad. Sci. U.S.A. 89:2804-8; Lansdorp, P.M., H. J. Sutherland et al., (1990) “Selective expression of CD45 isoforms on functional subpopulations of CD34+ hematopoietic cells from human bone marrow.” J. Exp. Med. 172:363-6; Sato, N., K. Sawada et al., (1991) “Purification of human marrow progenitor cells and demonstration of the direct action of macrophage colony-stimulating factor on colony-forming unit-macrophage” Blood 78:967-74; Smith, C., C. Gasparetto et al., (1991) “Purification and partial characterization of a human hematopoietic precursor population” Blood 77:2122-8; Udomsakdi, C., C. J. Eaves et al., (1991) “Separation of functionally distinct subpopulations of primitive human hematopoietic cells using rhodamine-123” Exp. Hematol. 19:338-42; Udomsakdi, C., P. M. Lansdorp et al., (1992) “Characterization of primitive hematopoietic cells in normal human peripheral blood” Blood 80:2513-21.

The treated mononuclear cells and hematopoietic cells are typically administered to the recipient in a pharmaceutically acceptable carrier by intravenous infusion. Carriers for these cells can include but are not limited to solutions of phosphate buffered saline (PBS) containing a mixture of salts in physiologic concentrations.

The following examples are put forth so as to provide those of ordinary skill in the art with a complete disclosure and description of how the compositions and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. The present invention is more particularly described in the following examples which are intended as illustrative only since numerous modifications and variations therein will be apparent to those skilled in the art.

### EXAMPLE 1

Animals:

B10.BR (H2^{k}), DBA/2 (H2^{d}), Balb/C (H2^{d}), CD45.1/CD45.2 congenic strains of C57.BL6 (H2^{b}) mice, and a C57.BL6 mutant strain having defective NK cells (beige), aged 8 to 10 weeks, were purchased from Jackson Laboratories (Bar Harbor, Me.). A congenic strain of C57.BL6 (H2b) expressing Thy 1.1 was obtained from Miriam Lieberman (Stanford University, CA) and bred at Emory. Mice were maintained in micro-isolator cages in the Emory University Animal Care Facility, with acidified water and standard chow available at all times. Animal handling and experimental procedures were in concordance with the Guide for the Care and Use of Laboratory Animals published by the National Academy Press, Washington, D.C., 1996, and approved by the Emory University Institutional Animal Care and Use Committee (IACUC).

T-Cell Depleted Bone Marrow (TCD-BM):

Femora and tibiae were removed and BM cells expelled by flushing sterile Hank's balanced salt solution containing 3% heat-inactivated fetal bovine serum (HBSS/FBS) through the shaft using a 25-gauge needle and syringe. Harvested cells were incubated with biotinylated anti-CD3 or anti-CD5 antibody (Pharmingen, San Diego, CA) at saturating concentration, washed, and then incubated with Streptavidin Microbeads (Miltenyi Biotech, Gmbh). The cell suspensions were then passed through a Vario MACS magnetic separation column (Miltenyi Biotech) to immunomagnetically deplete the BM of T-cells. The TCD-BM was routinely analyzed by flow cytometry, and always contained <1% residual T-cells.

Fludarabine-Treated Splenocytes:

Splenocytes were harvested by perfusing spleens with sterile HBSS/FBS, and washed before culture. Splenocytes (10×10⁶ cells/ml) were incubated in complete media (RPMI supplemented with 10% heat-inactivated FBS, 50 μM 2-mercaptoethanol, 100 Units/ml penicillin, 100 ug/ml streptomycin, 292 μg/ml L-glutamine, 1 mM sodium pyruvate, and non-essential amino acids added according to the manufacturer's specifications (Biowhittaker, Walkersville, Md.)). Fludarabine (Berlex Laboratories, Richmond, Calif.) was added to a final concentration of 20 μg/ml; control splenocytes were cultured without fludarabine. Fludarabine treated and control cultures were maintained at 37° C. in a 5% CO₂ incubator for 24 hours. Overall viability was assessed by counting ethidium bromide/acridine orange stained cells under fluorescence microscopy. Survival of T-cell populations was determined by flow cytometric analyses using fluorochrome conjugated antibodies against mouse CD3, CD4 and CD8. Propidium iodide stain was used to exclude dead cells from the analysis. In all subsequent transplant and in vitro experiments, the number of fludarabine treated or untreated splenocytes used was based on the number of VIABLE cells in the culture after 24 hour incubation.

Conditioning and Transplant:

Recipient mice were irradiated 1 day prior to transplant using a ¹³⁷CS source at a rate of 1.24 Gy/min. Two doses of 5.5 Gy were administered, 3 hours apart, for a total lethal dose of 11 Gy. Irradiated mice were given drinking water containing antibiotics (1.1 mg/mL neomycin sulfate and 1,000 U/mL polymixin sulfate) for 4 weeks following BMT. Cell suspensions for transplant were prepared in 0.2 mL HBSS/FBS. For GvHD and graft facilitation experiments, 1×10⁶ TCD-BM cells were administered alone or mixed with 1×10⁷ viable fludarabine-treated or untreated splenocytes removed from 24 hr culture. Some experiments also used mixtures of TCD-BM and irradiated (7.5 Gy) splenocytes. All transplants were done via tail vein injection. The mice were monitored daily for survival; moribund animals were euthanized. Surviving animals were weighed to monitor progression of GvHD. Weights were recorded twice a week for the first month following transplant, and then weekly until termination of the experiment.

Leukemic Cell Lines and GvL Transplant Experiments:

The C1498 myeloid leukemia (C57.BL6 congenic) and LBRM T-cell lymphoma (B10.BR congenic) cell lines were obtained from American Type Culture Collection (Rockville, Md.), and used here as transplantable leukemic tumors in GvL experiments. Recipient mice were irradiated on Day −2, administered congenic tumor cells via tail vein injection on Day −1, and transplanted with allogeneic TCD-BM and splenocytes on Day 0. Depending on the experimental protocol, additional infusions of splenocytes were administered on a weekly basis as indicated.

Analysis of Hematopoietic Chimerism:

Peripheral blood (0.2 ml) was collected by tail vein nick at 1 to 4 months post-BMT. Red blood cells (RBC) were depleted by 1G sedimentation using 3% Dextran T500 in HBSS. Residual RBC were removed by hypotonic saline lysis¹¹. Donor and host-derived leukocytes and T-cells were distinguished by FACS analysis using fluorochrome-conjugated monoclonal antibodies specific for H2^{b} and H2^{k} MHC, as well as specific leukocyte markers (CD3, CD4, CD8, Thy 1.1, Thy 1.2, CD45.1 and CD45.2; Pharmingen). Some experiments also incorporated white blood cell counts (obtained using Baker Instruments System 9000, Allentown, Pa.).

Proliferative Response to Allo-Antigen (Mixed Lymphocyte Reaction, MLR) and Mitogen:

4×10⁶ cells/ml responder cells (either B10.BR, DBA/2, or Balb/c) were co-cultured with 2×10⁶ irradiated (25 Gy) stimulator cells/ml (C57.BL6) in complete media with 10 U/ml IL-2 (Chiron Corporation, Emeryville, Calif.) and 10 ng/ml IL-7 (PeproTech Inc., Rocky Hill, N.J.) in 96 well plates. Proliferation was monitored by measuring DNA synthesis/thymidine incorporation. Cultures were pulsed with 1 uCi/well of tritiated thymidine (NEN Life Sciences, Boston, Mass.) on day 6, harvested onto filtermats using a Tomtec Harvester (Hamden, Conn.) on day 7, and read on MicroBeta TriLux counter (PerkinElmer, Inc., Gaithersburg, Md.).

Limiting Dilution Analysis:

The frequency of alloreactive cells was measured using a limiting dilution assay . Briefly, 20,000 responder cells were three-fold serially diluted in 96-well plates with 12 replicate wells per dilution. 10,000 stimulator cells were added to each well in a combined total volume of 150 μl complete media. After 7 days in co-culture, the proliferation of responder cells was measured using tritiated thymidine incorporation. The number of responder cells was plotted versus the natural logarithm of the percentage of negative wells (i.e., wells with cpm readings lesser than media control). The reciprocal of the slope of the regression line yielded the frequency of alloreactive T-cells.

Cytotoxic T Lymphocytes (CTLs):

A cell plating protocol similar to the MLR was initially done. On day 7, the co-culture was split and re-stimulated with 0.5×10⁶ cells/ml irradiated stimulators. Additional IL-2 was added for a final concentration of 100 U/ml while IL-7 was maintained at 10 ng/ml. A standard 4-hour chromium release assay was used to measure the cytotoxic activity of the generated CTLs¹³. 1×10⁶ C1498 leukemia cell targets (T) were labeled overnight with 100 uCi sodium chromate (⁵¹Cr) (NEN Life Science Products) in 1 ml complete media. Labeled targets were washed 2× with 1% FBS/RPMI to rinse excess ⁵¹Cr. CTL effector (E) cells were serially diluted in 96 well plates and incubated with labeled targets to make-up desired E:T ratios. Spontaneous and maximum isotope release were determined by incubation of labeled target cells in complete media or 2% Triton X-100, respectively. After a 4 hour incubation period, the plates were centrifuged and 50 μl supernatant was transferred to a plastic 96 well round bottom plate containing 150 μl per well of Optiphase scintillation liquid (Wallac). The plate was sealed with plastic top cover (Excel Scientific, Wrightwood, Calif.) and read using a MicroBeta TriLux counter (Wallac). Target specific lysis was calculated by the formula: %cytotoxicity=(cpmₜₑₛₜ−cpmₛₚₒₙₜₐₙₑₒᵤₛ)/(cpmₘₐₓᵢₘᵤₘ−cpmₛₚₒₙₜₐₙₑₒᵤₛ).

Statistical Analysis.

Kaplan-Meier analysis was used to analyze survival of the animals. The differences between survival among different treatment groups were tested using the Wilcoxon log rank test¹⁴. Student t-test was used to compare effects of fludarabine in the in vitro assays.

Ex vivo fludarabine treatment resulted in reduction of T-cell and overall leukocyte viability: Initial experiments were conducted to determine the recovery of viable splenocytes after 24hr culture in media containing graded doses of fludarabine (0, 3, 10, 20 or 40 μg/ml), and to characterize the GvHD potential of the recovered splenocytes in the BL6 versus B10.BR transplant model. Increasing loss of viability among cultured cells was dose dependent to 10 μg/ml fludarabine, with a plateau seen at higher fludarabine doses. Cultures treated with 20 μg/ml fludarabine for 24hr had approximately 42% overall cell viability, while control cultures had ˜75% viability compared to the starting cell population. Viability of T-cells was reduced after fludarabine or control culture, although to a lesser extent than for the bulk splenocytes (˜60-70% T-cell viability after fludarabine culture and 80-85% T-cell viability after control culture, Table 1). A dose of 20 μg/ml was selected for further studies since it resulted in a consistent effect on viability after 24 hr culture, and pilot allogeneic transplant experiments to test GvHD reduction demonstrated that mice receiving TCD-BM plus splenocytes treated with 20 μg/ml fludarabine had the most favorable 40 day survival rate compared to groups receiving control splenocytes or splenocytes treated with other doses of fludarabine. Note that for all subsequent transplant and in vitro experiments, the number of fludarabine treated or untreated splenocytes used was based on the number of VIABLE cells in the culture after the 24 hour incubation period.

Treatment of Allogeneic Lymphocytes with Fludarabine Reduced GvHD and Facilitated Engraftment in the MHC Mis-Matched C57.BL6→B10.BR Transplant Model.

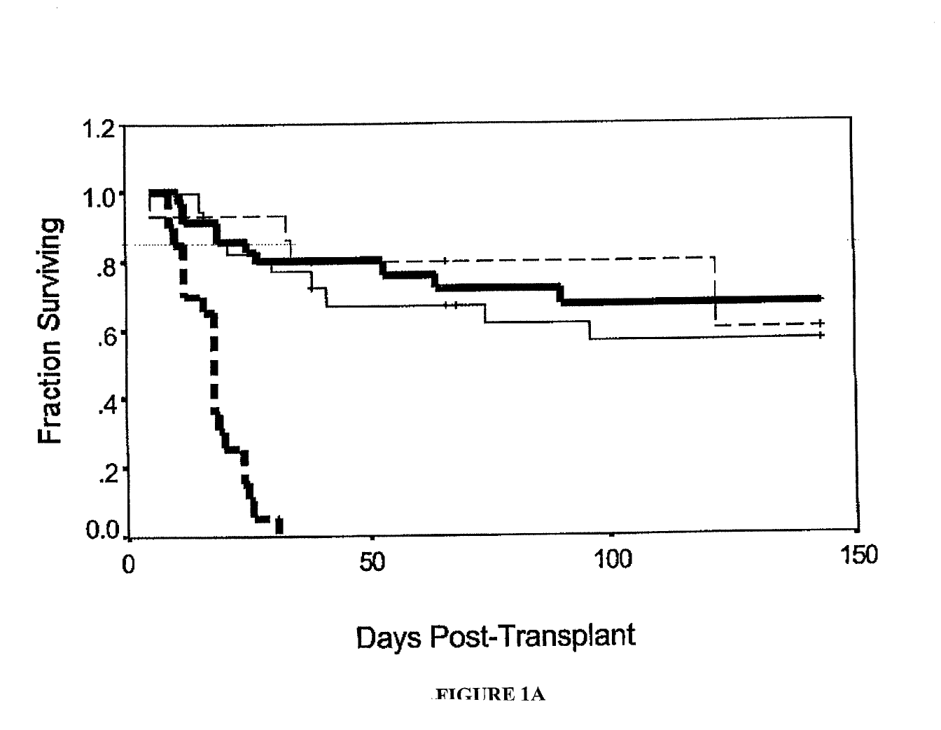

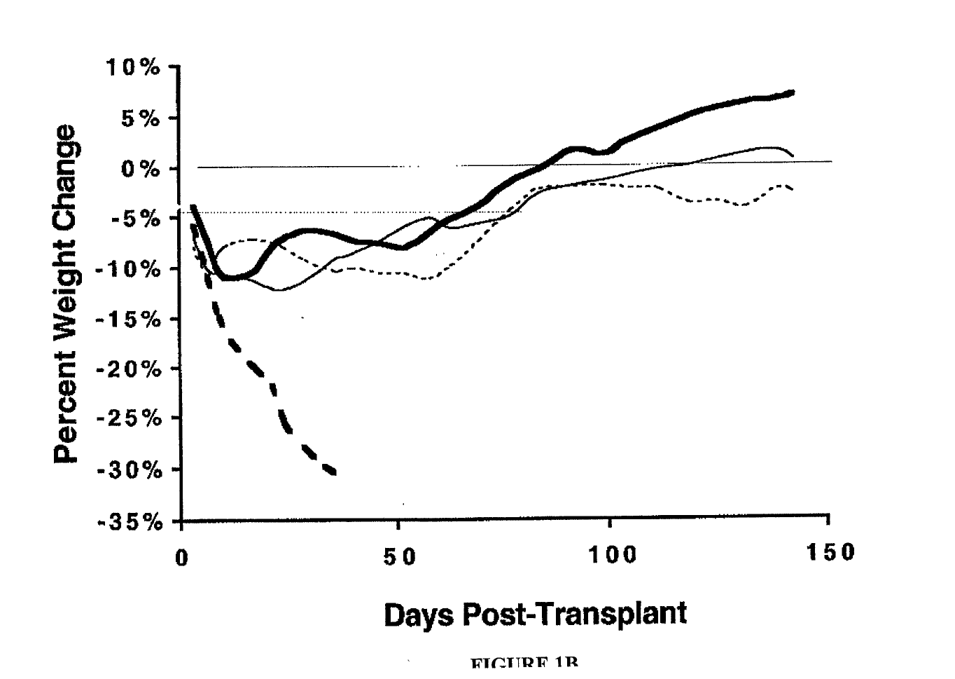

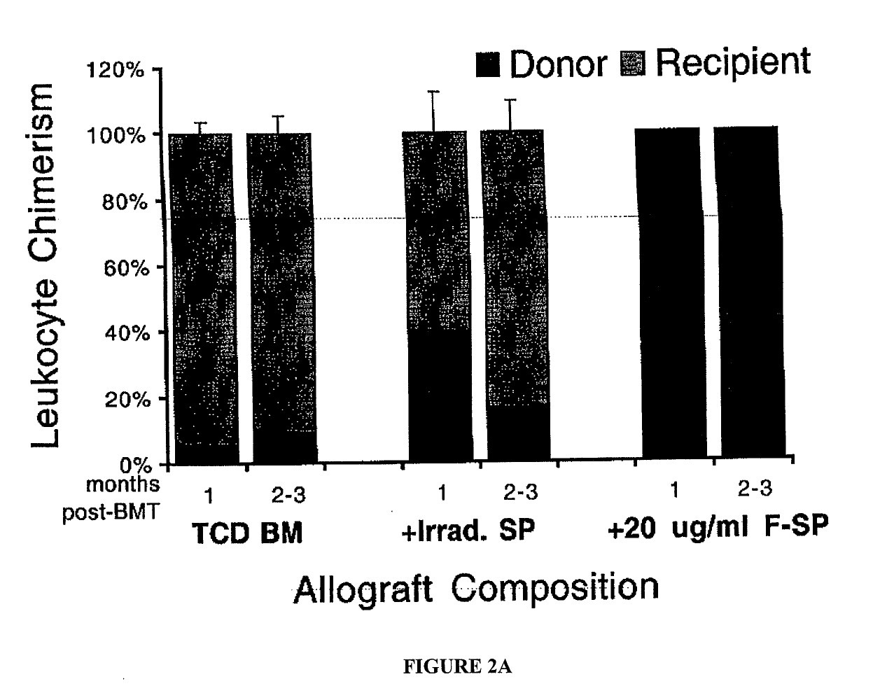

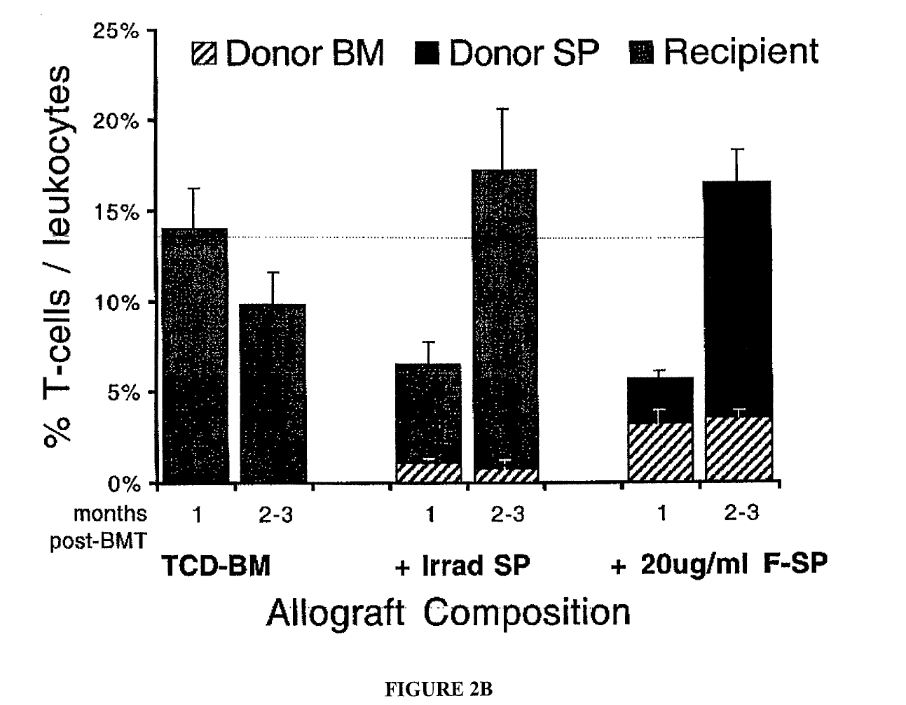

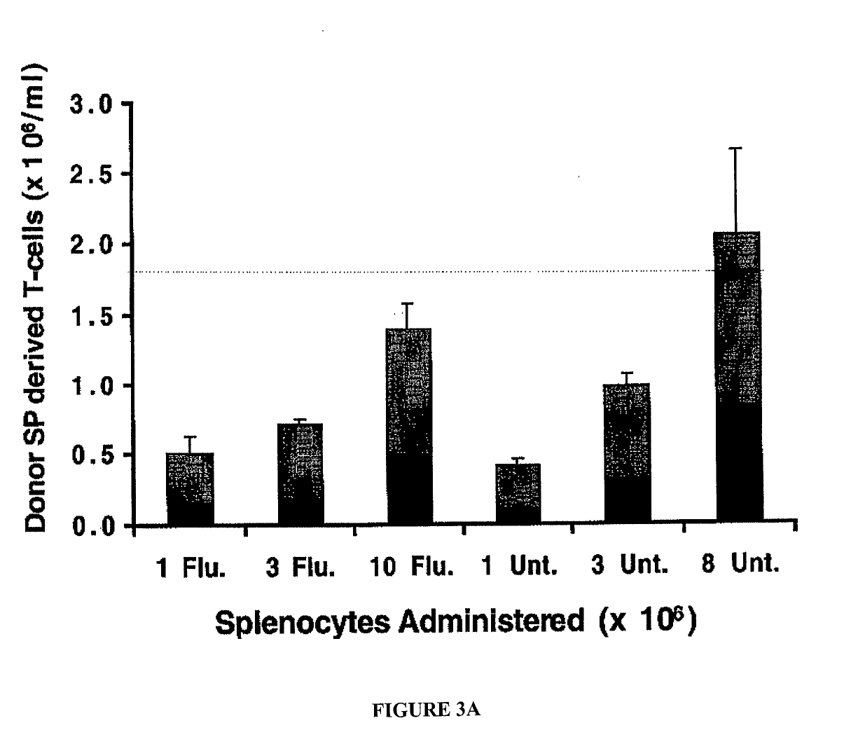

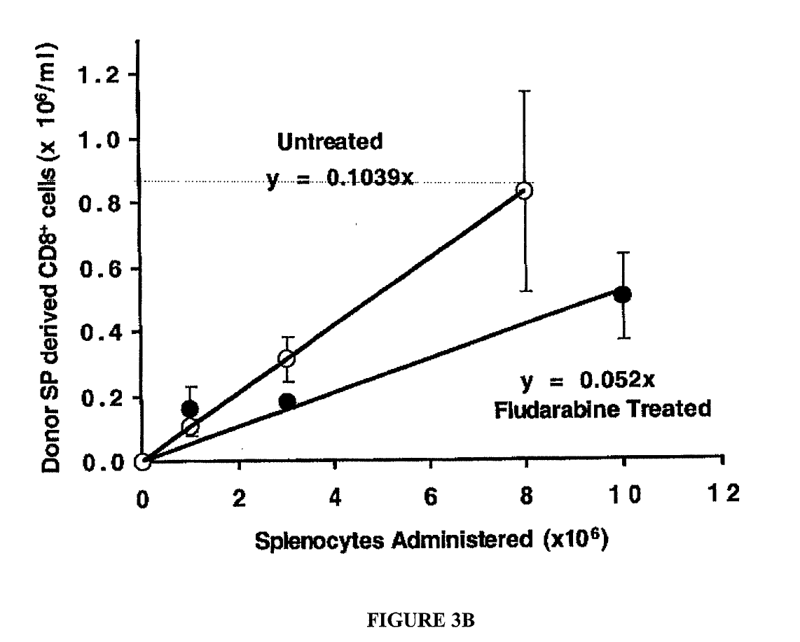

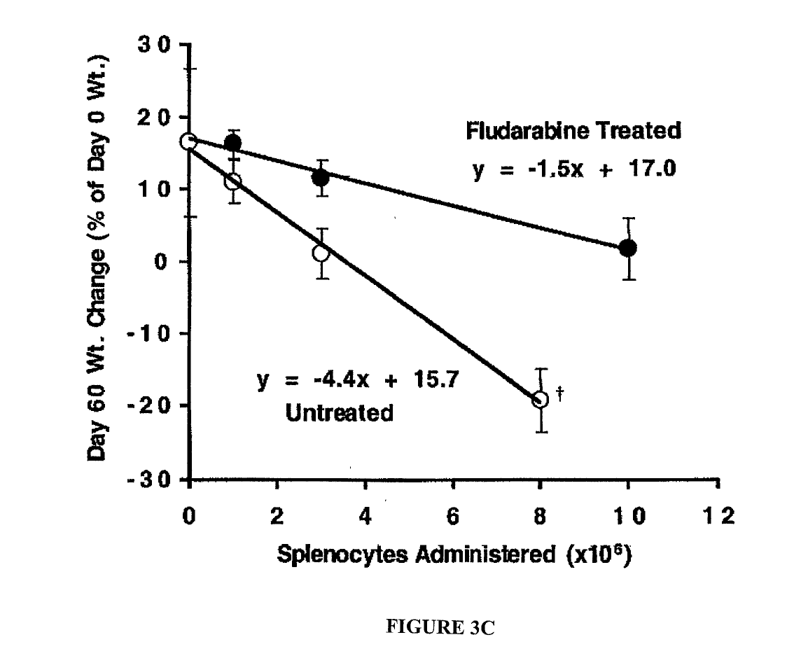

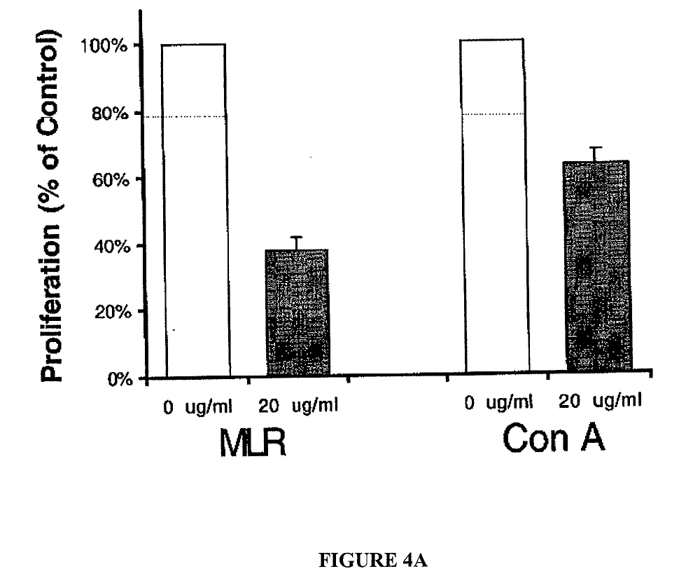

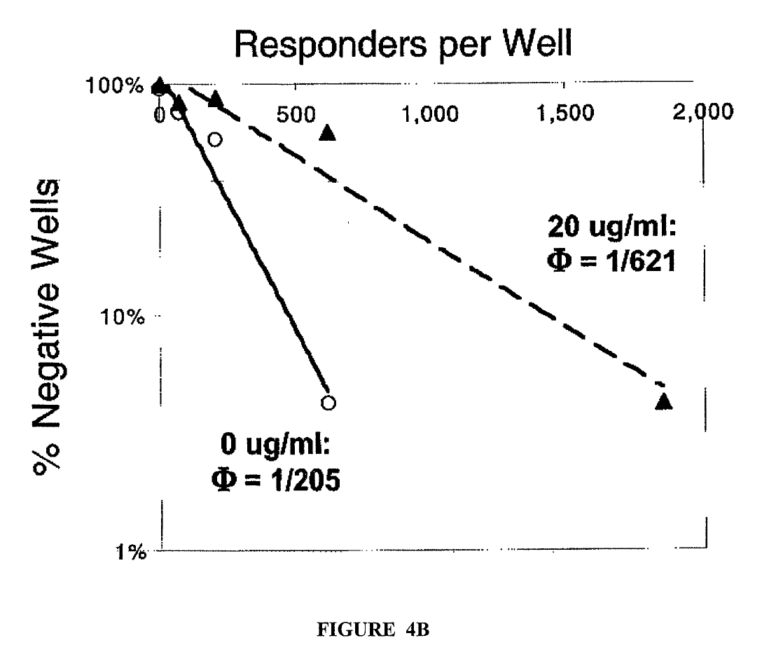

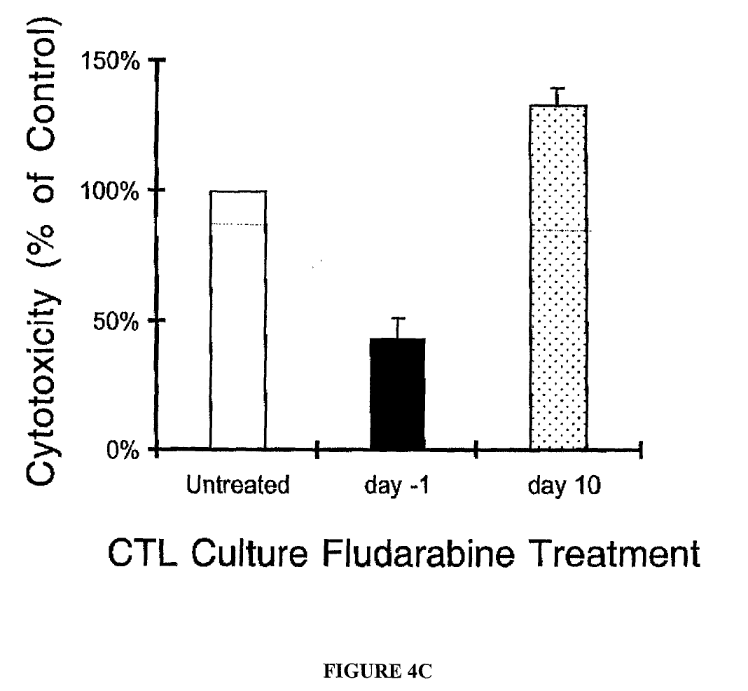

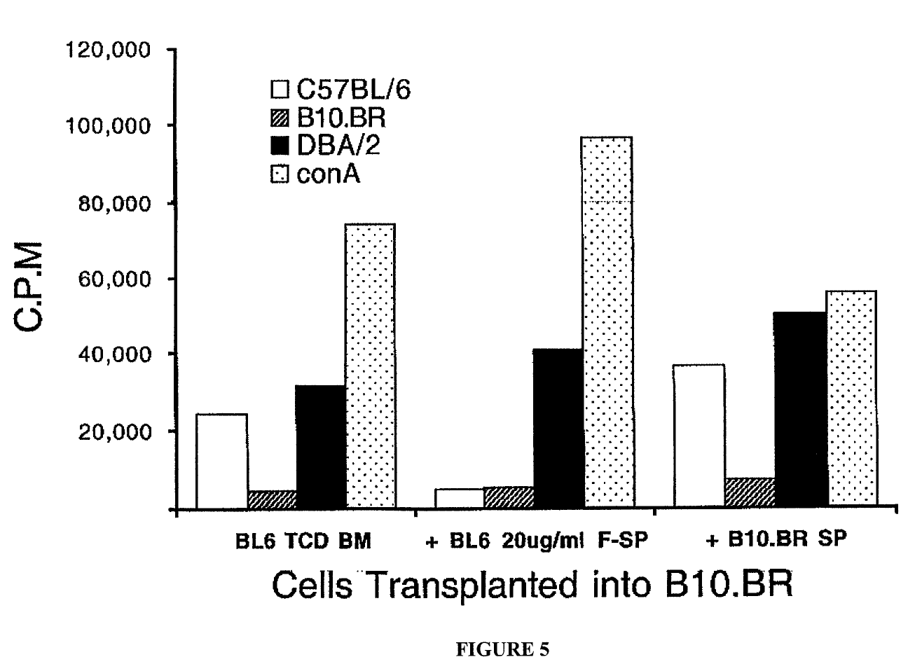

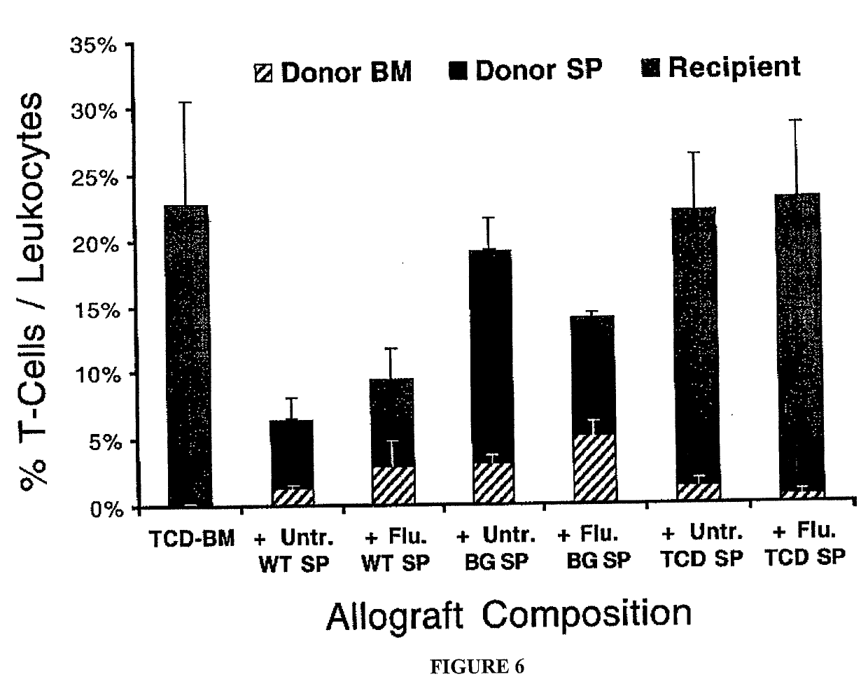

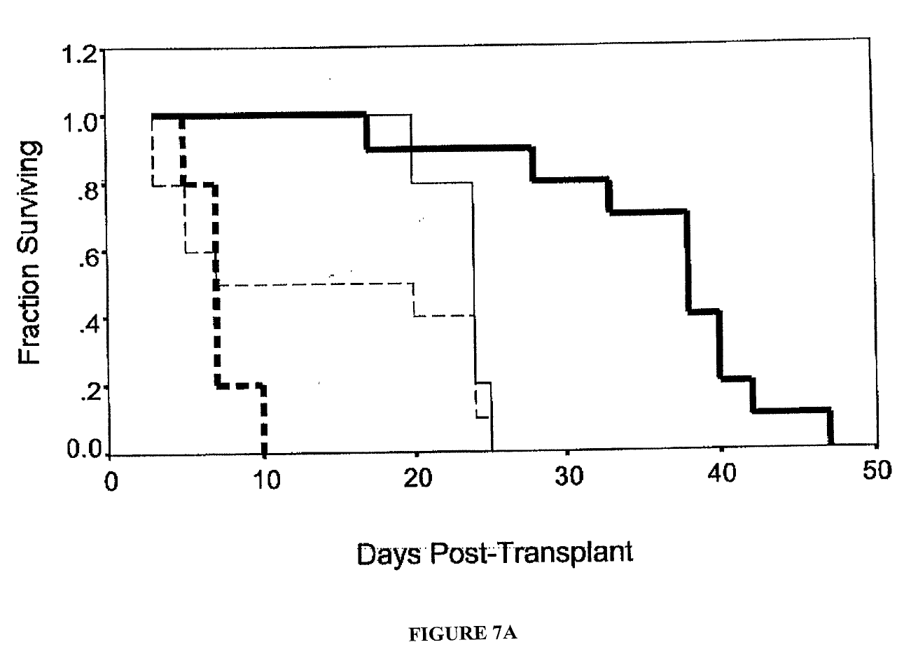

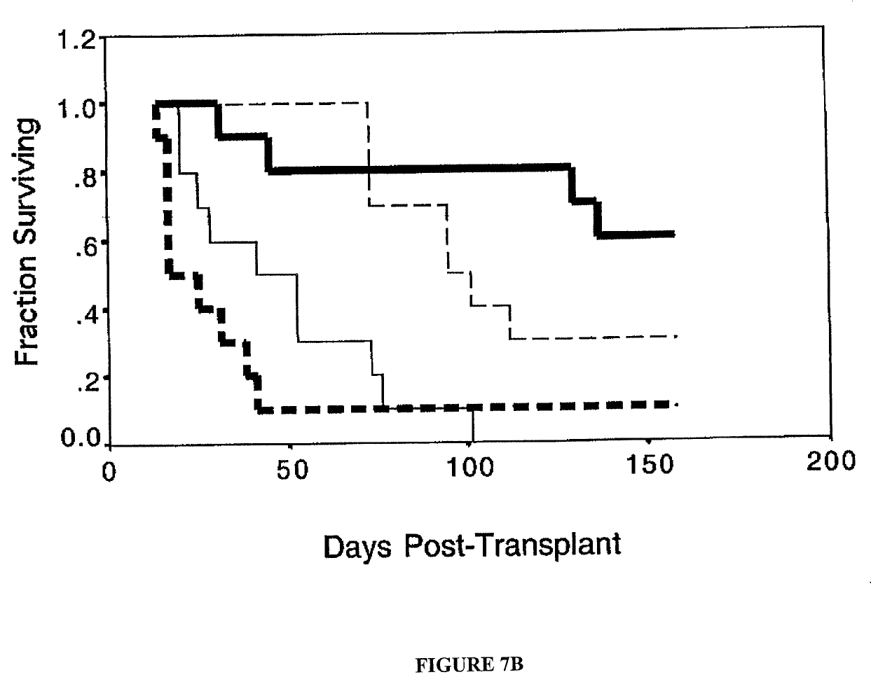

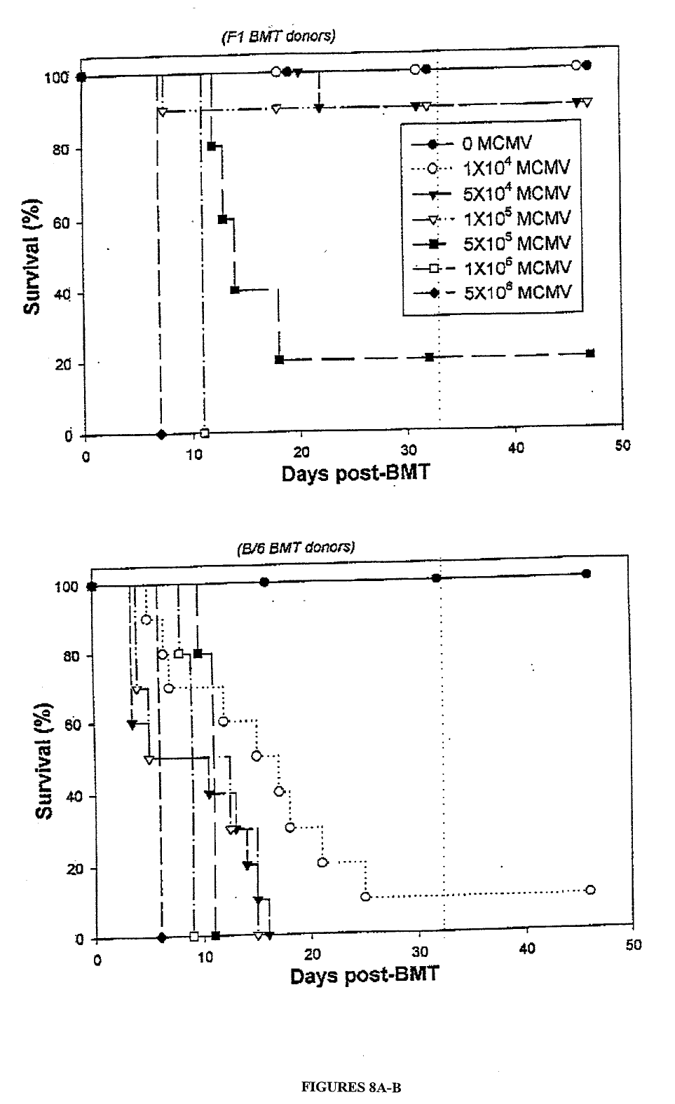

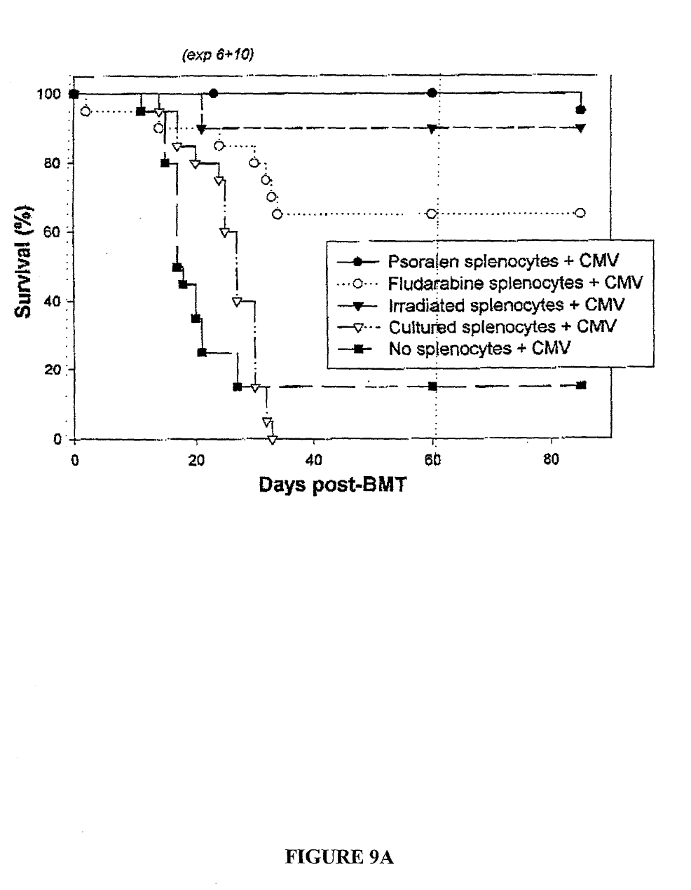

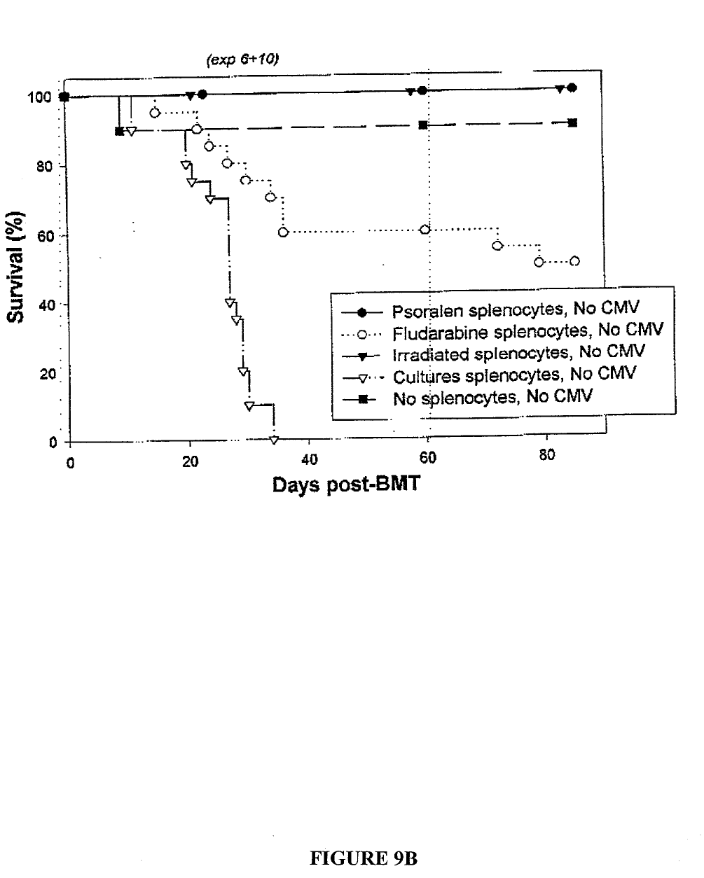

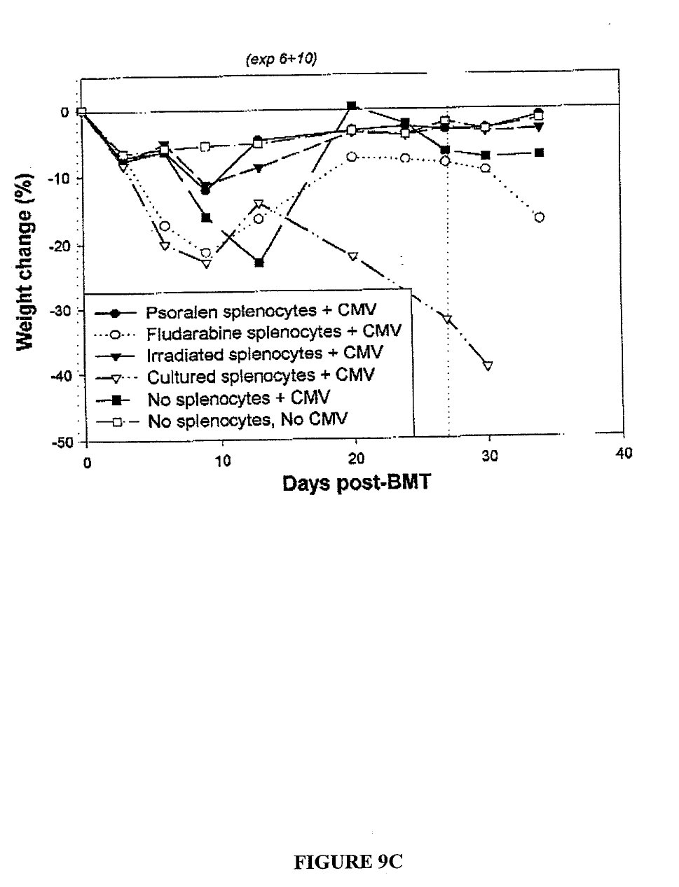

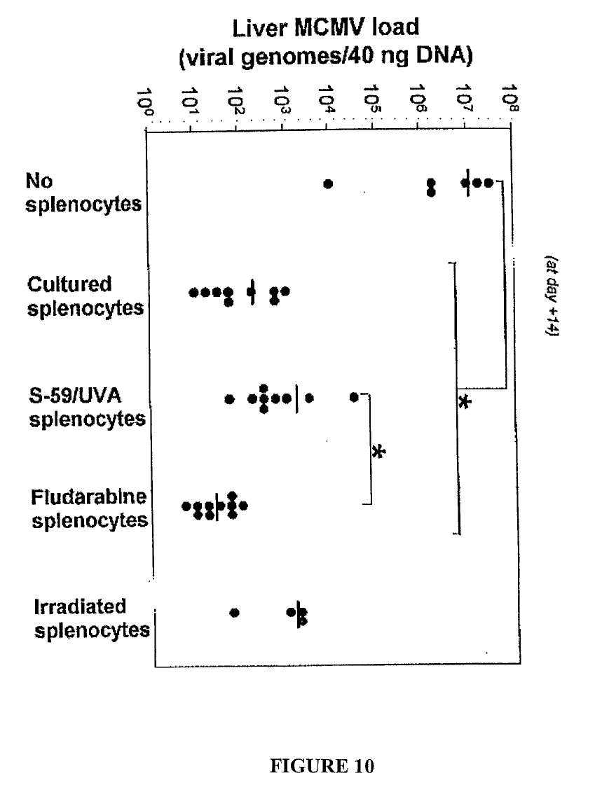

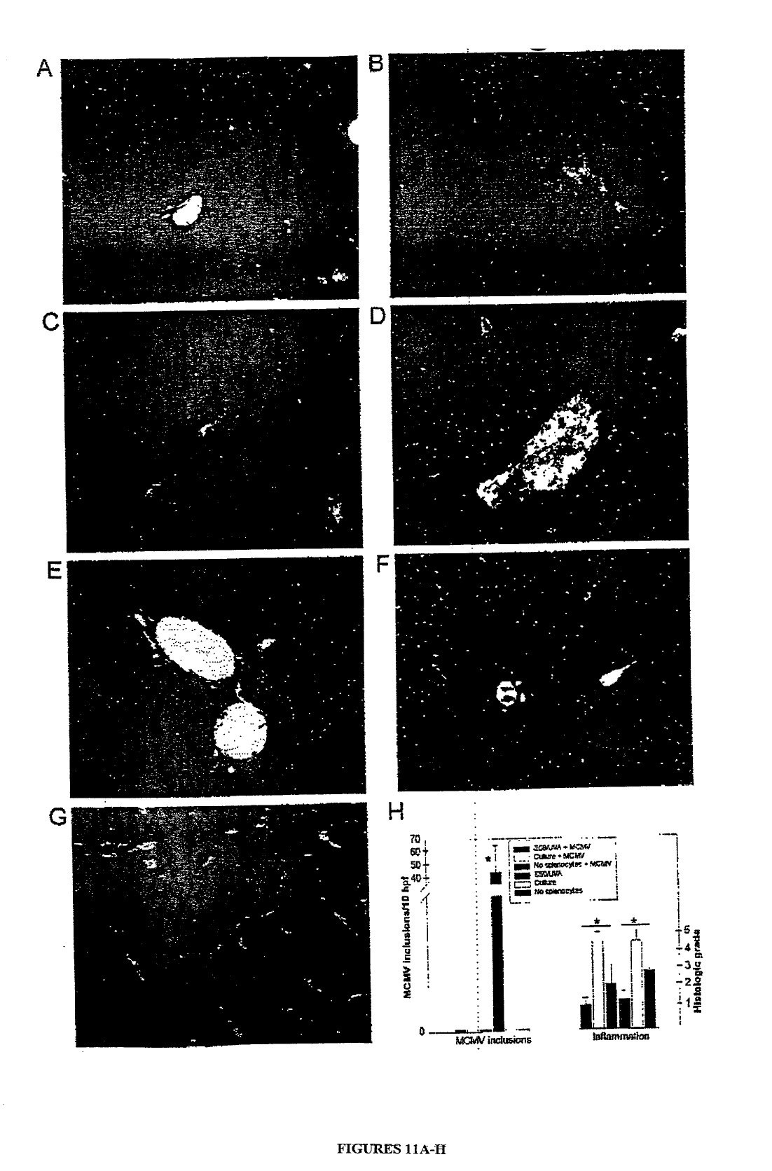

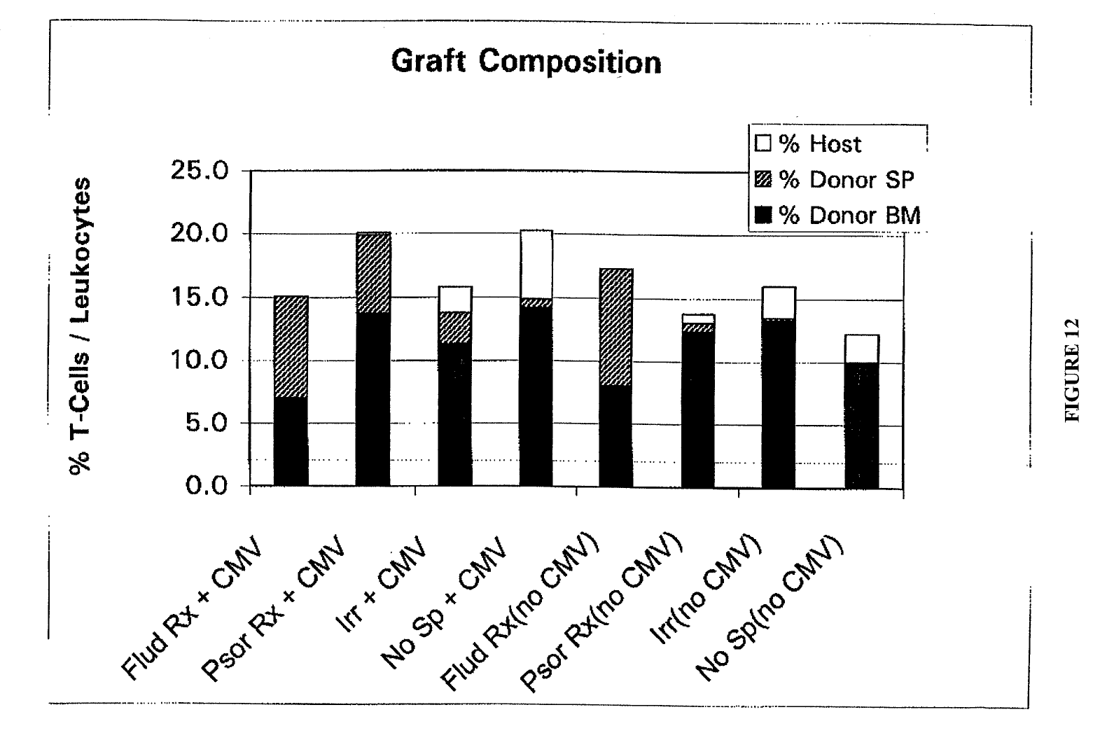

Lethally irradiated B10.BR mice transplanted with 1×10⁶ allogeneic (BL6) TCD BM cells had a long term survival of ˜60% (measured at 140 days, Figure la). Mortality among recipients of TCD allogeneic BM occurred secondarily due to graft failure rather than GvHD¹, with autologous hematopoietic reconstitution noted among surviving mice (FIG. 2A). Co-transplantation of 1×10⁷ allogeneic splenocytes cultured for 24 hr in media alone (control splenocytes) resulted in acute GvHD manifested as severe weight loss (FIG. 1B), and rapid mortality (FIG. 1A). When allogeneic TCD-BM was co-transplanted with 6×10⁷ irradiated (7.5 Gy) allogeneic splenocytes, the GvHD effect was mitigated. Mice which received irradiated allogeneic splenocytes had a survival rate of nearly 60% at 140 days, and the body weight of surviving mice returned to normal over this time (FIG. 1B). However, there was only limited improvement in overall donor-derived hematopoietic reconstitution compared to mice receiving TCD BM only (FIG. 2A). The results obtained after co-transplantation of allogeneic TCD BM and irradiated splenocytes recapitulate previous findings¹.

B10.BR mice which received BL6 TCD-BM plus 1×10⁷ viable fludarabine-treated BL6 splenocytes (20 μg/ml, 24 hr culture) had ˜65% survival at 140 days post-transplant, significantly improved compared to survival among recipients of TCD-BM plus control splenocytes, p<0.0005, FIG. 1A). Post-transplant weight recovery indicated an absence of chronic GvHD among the surviving animals (FIG. 1B). The most striking observation was the rapid and durable donor-derived hematopoietic reconstitution, measured in peripheral blood at 1 and 2-3 months post-transplant (FIGS. 2A, 2B). Recipients of allogeneic TCD-BM plus fludarabine-treated splenocytes had >99% donor cells in their peripheral blood measured at 1 month and 2-3 months post-transplant (FIG. 2A). The relative contribution of donor TCD-BM cells and splenocytes to peripheral T-cell reconstitution was assessed using donor strains expressing different Thy 1.1/Thy 1.2 isoforms (FIG. 2B). For groups receiving fludarabine-treated splenocytes, T-cells represented ˜6% of blood leukocytes at 1 month post-transplant, with equal contribution from donor BM and splenocytes. The splenocyte-derived fraction was expanded at 2-3 months post-transplant, bringing the total T-cell compartment to ˜15% of blood leukocytes. In contrast, mice which received TCD-BM plus irradiated splenocytes did not have significant numbers of donor splenocyte-derived T-cells in their blood, while TCD-BM derived T-cells comprised ˜1% of leukocytes, and the remainder of T-cells were derived from autologous reconstitution (˜5% at 1 month post-transplant and ˜16% at 2-3 months post-transplant, FIG. 2B).

Fludarabine Treatment Improved the Engraftment/GvHD Activity Ratio of Allogeneic Donor Splenocytes.

In order to determine the relative engraftment potentials and GvHD activities of untreated and fludarabine treated allogeneic donor splenocytes, transplant experiments were conducted using 1×10⁶ TCD-BM cells combined with 1, 3, or 10×10⁶ fludarabine treated splenocytes, or 1, 3, or 8×10⁶ untreated splenocytes. The inclusion of animals transplanted with lower numbers of splenocytes than used in a standard model (10×10⁶) allows comparison of the activities of fludarabine treated cells to untreated cells at doses which do not cause rapid GvHD related mortality. TCD-BM and splenocytes from BL6 strains having different Thy1.1 or Thy1.2 isotypes were administered to lethally irradiated B10.BR mice. 20 mice were transplanted per group, except the groups which received TCD-BM alone (n=5), or TCD-BM+8×10⁶ untreated splenocytes (n=10). An additional 10 mice received TCD-BM+10×10⁶ untreated splenocytes; as expected, these mice died rapidly and were not included in the following analyses. All groups that received donor splenocytes had 100% donor-derived peripheral blood leukocytes at 60 days post-transplant. The contribution of donor TCD-BM and splenocyte-derived cells to the peripheral blood T-cell population was determined using flow cytometry and blood cell counts at 2-4 months post transplant. The donor splenocyte derived fractions of CD4+ and CD8+ cells are shown in FIG. 3A; for both untreated and fludarabine treated splenocytes, the contribution from these sources increased with the number of cells administered, and was associated with decreased survival as described in the figure legend. In order to determine the relative T-cell engraftment potential, the donor splenocyte derived CD8+cell fraction was plotted against splenocyte dose (FIG. 3B). By comparing the slopes of the lines determined in this manner, fludarabine treated splenocytes were determined to have half the engraftment potential (m=0.052) of untreated splenocytes (m=0.1039). Similar results were obtained for CD4+ cell engraftment. The relative GvHD activity was determined by plotting the weight change at day 60 post-transplant against the number of splenocytes administered (FIG. 3C). The slope of the line determined for fludarabine treated splenocytes (m=-1.5) was approximately one third of that determined for untreated splenocytes (m=-4.4), indicating that fludarabine treated splenocytes have one third the GvHD activity of untreated splenocytes. Taken together, the data demonstrate a 50% improvement in the T-cell engraftment/GvHD activity ratio for fludarabine treated splenocytes, compared to untreated splenocytes.

Fludarabine Treated T-Cells had Decreased Proliferative Response to Allo-Antigen in vitro.

The proliferative capacity of the T-cells that survived fludarabine exposure was assessed in a mixed lymphocyte reaction (MLR). Prior exposure to 20 μg/ml fludarabine for 24 hours resulted in a 62% reduction of thymidine incorporation after stimulation with allogeneic antigen (p=0.0006, FIG. 4a). Reductions in the proliferative response to concanavalin A was less pronounced (37% decrease compared to control, p=0.0 15, FIG. 4a). The reduced proliferation in vitro was concomitant with a 66% decrease in the frequency of allo-reactive T-cells measured by limiting dilution assay (1/205 alloreactive cells in untreated culture, 1/621 alloreactive cells in fludarabine treated culture; FIG. 4b). Cytotoxic T-cells (CTL's) generated using splenocytes treated with fludarabine (20 μg/ml, 24hr) prior to allo-stimulation exhibited a 57% reduction in cytotoxicity when compared with CTL's generated using T-cells incubated for 24hr in control culture prior to initiation of a MLR (p<0.05, FIG. 4c). CTL's were generated using Balb/C (H2^{d}), DBA/2 (H2^{d}) or B10.BR (H2^{k}) splenocytes allo-stimulated with irradiated C57.BL6 (H2^{b}) splenocytes. C1498 leukemia cells (C57.BL6 derived, H2^{b}) served as the target cells in cytotoxicity assays. Interestingly, the viability of predominantly CD8+ cultured CTL's was not affected by fludarabine in contrast to the effect of the drug on naive spleen cells (Table 1), suggesting different cytolytic mechanisms of fludarabine to resting and activated lymphocytes. Cytotoxicity of allo-primed CTL's treated with fludarabine on day 10 of an MLR was not significantly different from control non-treated allo-stimulated T-cells (FIG. 4c). These observations suggest that fludarabine treatment does not directly inhibit the cytotoxic effector mechanisms of activated CTL but may lead to immuno-modulation of alloreactive cells during subsequent allo-antigen exposure.

The ability of splenocytes from donor-engrafted recipients to generate an allo-response to the original host (H2^{k}), donor (H2^{b}), or a third party (H2^{d}) type antigen was assessed. Spleen cells from transplant recipients of allogeneic TCD-BM plus fludarabine-treated splenocytes and recipients of allogeneic TCD-BM alone were harvested 8 months post-BMT. Splenocytes were cultured with irradiated spleen cells from original donor and recipient-type as well as third-party (DBA/2, H2^{d}) type mice. The proliferative responses of transplanted recipients were measured in a 7-day MLR assay. Recipients of TCD BM alone had graft rejection with autologous reconstitution; hence the recipients' T-cells remained non-reactive to host antigen and reactive to donor and third-party allo-antigen (FIG. 5). Splenocytes from mice which received allogeneic TCD-BM and fludarabine-treated splenocytes were tolerant to both host and donor type allo-antigen but had normal proliferative reactivity to third party allo-antigen and mitogen (FIG. 5), indicating that prior fludarabine exposure had not produced a global effect on the proliferative responses of donor-derived T-cells.

Donor T-Cells in Fludarabine-Treated Splenocyte Infusions were Necessary for Graft Facilitation.

The role of T- and NK-cells in graft facilitation was studied in MHC mis-matched transplants using the C57.BL6_B10.BR strain combination, with additional groups that received T-cell depleted splenocytes or splenocytes from beige mutants (FIG. 6). At 27 days post-transplant, recipients of TCD-BM alone had autologous reconstitution, as shown earlier (FIG. 2a), with negligible donor-derived T-cells. Recipients of TCD-BM plus control or fludarabine-treated splenocytes from WT allogeneic donors had >5% donor-derived T-cells among blood leukocytes, suggesting graft facilitation mediated by the splenocyte infusions (FIG. 6). Although the levels of T-cell chimerism at day 27 were similar, survival and GvHD (weight) data show that recipients of fludarabine-treated splenocytes have a marked survival advantage over mice which received untreated splenocytes (FIG. 1a, 1b). Recipients of TCD splenocytes that were fludarabine treated or cultured in media alone had mixed T-cell chimerism, with a greatly increased number of recipient-derived T-cells in the blood, similar to mice which received TCD-BM alone. Taken together, these data support the requirement of donor T-cells for graft facilitation using fludarabine treated allogeneic splenocytes. The T-cells of donor splenocyte derivation observed among recipients of TCD splenocytes may be attributable to expansion of residual T-cells in the TCD splenocyte graft. In contrast, mice which received splenocytes from beige mutants that were untreated or fludarabine treated had >15% donor-derived T-cells among blood leukocytes, indicating that NK cells in the donor splenocyte infusion were not necessary for graft facilitation. The increased numbers of donor-derived T-cells among recipients of untreated or fludarabine-treated beige splenocytes may reflect a difference in the activation status of T-cells from the mutant strain compared to wild type T-cells.

Fludarabine-Treated Splenocyte Infusions have Superior GvL Activity Compared with Irradiated Allogeneic Splenocytes.

C57.BL6 mice were lethally irradiated then transplanted with a lethal cell number (2×10⁵) of a congenic myeloid leukemia cell line C1498, and TCD B10.BR BM (1×10⁶ cells). The anti-leukemic activity of fludarabine treated (20 μg/ml), irradiated (7.5 Gy) or control B10.BR splenocytes were compared using this model system. Recipients of fludarabine-treated splenocytes had significantly improved survival compared to either recipients of TCD BM alone, a combination of TCD BM and irradiated splenocytes, or TCD BM and untreated splenocytes (p<0.005, FIG. 7a). However, the recipients of a single injection of fludarabine treated splenocytes eventually succumbed to leukemia, indicating that a single infusion of fludarabine treated cells was insufficient to completely eradicate this rapidly growing myeloid leukemia cell line (FIG. 7a). In order to investigate the anti-tumor potential of fludarabine treated donor splenocytes against a inore indolent lymphoma, the lethality of LBRM, a T-cell lymphoma¹⁵ derived from B10.BR mice was established. B10.BR mice injected with 3×10⁵ to 3×10⁶ LBRM cells died of lymphoma after 60-90 days. The anti-leukemic activity of irradiated or fludarabine treated splenocytes was then studied in B10.BR transplant recipients which received a lethal dose (3×10⁶ cells) of LBRM. All recipient mice were conditioned with 11 Gy TBI (split fraction) on day −2, injected with LBRM on day −1, and transplanted with 1×10⁶ C57.BL6 TCD BM cells on day 0. One group received 5 weekly injections of 1×10⁷ fludarabine treated C57.BL6 splenocytes (days 0, 7, 14, 21, and 28). The second group received the same number and schedule of irradiated C57.BL6 splenocytes. The third group (control for GvHD) received untreated C57.BL6 splenocytes on the same schedule. The final group (control for the lethal effects of the LBRM lymphoma) received no post-transplant immunotherapy. Multiple weekly injections of fludarabine-treated splenocytes resulted in improved long-term survival among LBRM/transplant recipients compared to mice that received the combination of TCD-BM and LBRM without additional donor splenocytes (p=0.0004, FIG. 7b). In contrast, recipients of multiple injections of untreated splenocytes died rapidly of lethal GvHD (p=0.0012, compared to recipients of TCD-BM plus fludarabine treated splenocytes). Multiple injections of irradiated allogeneic splenocytes had a partial anti-leukemic activity compared to mice that received LBRM and TCD-BM alone (p=0.001 1). Thus, fludarabine-treated splenocytes have clear anti-leukemic activity against both myeloid and lymphoid malignancies in murine MHC mis-matched BMT, and recipients of single and multiple doses of fludarabine-treated splenocytes had limited GvHD, in contrast to recipients of untreated allogeneic splenocytes.

### EXAMPLE 2

Animals:

C57BL/6 (H2^{b})(CD45.2/Thy1.2), BALB/cJ (H2^{d})(CD45.2/Thy1.2), (C57BL/6×BALB/cByJ)F1(H2^{b/d}), and PepBoy (C57BL/6 (H2^{b}) CD45.1/Thy1.2) strains of mice aged 8-10 weeks were purchased from Jackson Laboratories (Bar Harbor, Me.). Bone marrow donor mice (BA-PepBoy: C57BL/6 (H2^{b}) CD45.1/Thy1.1) were bred by staff at the Emory University Animal Care Facility. Mice were given acidified sterile water and maintained in Micro Isolator cages (Lab Products Inc., Maywood, N.J.) at the Emory University Animal Care Facility. All experiments were performed in conformance with the Guide for the Care and Use of Laboratory Animals published by the National Academy Press, Washington, D.C. (1996), and approved by the Emory University Institutional Animal Care and Use Committee (IACUC).

Virus:

MCMV (Smith strain) was obtained from the American Type Culture Collection (Manassas, Va.; ATCC# VR-1399), and was subjected to three rounds of plaque-purification on monolayers of permissive NIH/3T3 cells (ATCC# CRL-1658) prior to use in experiments. NIH/3T3 cells were propagated in Dulbecco's modified Eagle's medium (DMEM) containing 4 mM L-glutamine, 1.5 g/L sodium bicarbonate, 4.5 g/L glucose, 100 IU/ml penicillin, 100 μg/ml streptomycin, and 10% calf serum (DMEM/CS). Cultures consistently tested negative for mycoplasma contamination (Mycoplasma PCR Primer Set; Stratagene). Sucrose gradient-purified MCMV stocks were prepared using published protocols (53), and stored in liquid nitrogen. Standard limiting dilution plaque assays using NIH/3T3 monolayers demonstrated titers ranging from 10^{7.1} to 10^{7.5} plaque-forming units (PFU)/ml, as calculated by the method of Karber (54).

Donor Bone Marrow Preparation:

Bone marrow (BM) was flushed from the femora and tibia of donor mice (F1 or BA-PepBoy) using sterile Hanks' balanced salt solution (HBSS) containing 3% heat-inactivated fetal bovine serum (HBSS/FBS). Harvested BM was incubated with biotinylated anti-CD3 or anti-CD5 antibodies (Pharmingen, San Diego, Calif.) at saturating concentration, washed once in 1% FBS/RPMI, and incubated with Streptavidin Microbeads (Miltenyi Biotech, Gmbh) in HBSS buffer. The cell suspensions were passed through a Vario MACS magnetic separation column (Miltenyi Biotech) to immunomagnetically deplete T-cells from the BM graft. Post-depletion analysis by flow cytometry routinely demonstrated <1% residual T-cells.

Preparation of Lymphocytes for Adoptive Immunotherapy:

Spleens were removed from either untreated PepBoy mice (naive donors), or those that had been inoculated with 10⁵-10⁶ PFU MCMV 2-4 months earlier (immune donors). Polyclonal splenocytes were harvested by perfusing spleens with sterile HBSS/FBS. Cells were washed, counted, and split into four separate cultures (fludarabine, gamma-irradiation, S-59/UVA, or untreated) at a density of 10×10⁶ cells/ml in complete media (CM: RPMI supplemented with 10% heat-inactivated FBS, 50 μM 2-mercaptoethanol, 100 U/ml penicillin, 100 μM g/ml streptomycin, 292 μg/ml L-glutamine, 1 mM sodium pyruvate, and non-essential amino acids added according to manufacturer's specifications (Biowhittaker, Walkersville, Md.)). All cultures were maintained in parallel for 24 hours at 37° C. in a 5% CO₂ atmosphere prior to adoptive transfer.

For fludarabine pretreatment, splenocytes in CM were incubated with fludarabine (Berlex Laboratories, Richmond, Calif.) at a final concentration of 20 μg/ml for the entire 24 hour culture period. To generate irradiated splenocytes for adoptive immunotherapy, cells were harvested after 24 hour culture and exposed to a single fraction of radiation from a ¹³⁷Cs source for a total of 7.5 Gy. S-59 psoralen (Cerus Corporation, Concord, Calif.) was added to some splenocytes cultures at the end of the 24 hour culture period at a final concentration of 2 nM. The cells in a T75 tissue culture flask were then placed on a UVA (320-400 nm) illumination device with a nominal fluorescence of 7 mW (Cole Parmer, Vernon Hill, Ill.) and illuminated for 5 mins to achieve 3.0 J/cm² equivalent UVA dose. Control (untreated) splenocytes were harvested after 24 hour culture without other interventions. For all four groups, lymphocytes were washed at the end of the culture/treatment period and the appropriate dosage of viable cells was used for adoptive immunotherapy.

Irradiation, Reconstitution, Adoptive Transfer, and MCMV Infection of BM Recipients:

One day prior to transplant, recipient F1 mice were conditioned using a ¹³⁷Cs source at a dose rate of 1.24 Gy/min. Two doses of 5.5 Gy were administered, 3 hours apart, for a total lethal dose of 11 Gy. Irradiated mice were given free access to drinking water containing oral antibiotics (1.1 mg/ml neomycin sulfate and 1000 U/ml polymyxin sulfate; Sigma, St. Louis, Mo.) starting 3 days prior to irradiation and continuing through the end of the experimental period. 5×10⁶ T-cell depleted (TCD) BM cells in a total volume of 0.2 ml HBSS/FBS were injected into the tail vein of F1 mice on the day following irradiation. In adoptive immunotherapy experiments, the appropriate number of donor splenocytes were mixed with TCD BM cells and injected simultaneously in 0.2 ml total volume. Two hours after BMT, mice were injected intraperitoneally with appropriate MCMV inocula diluted in DMEM/CS. Control (uninfected) mice received DMEM/CS alone. Mice were evaluated daily for morbidity and mortality. The development of clinical GvHD was prospectively monitored by weighing mice twice weekly, as well as observing for alopecia and/or ruffled fur, diarrhea, and decreased activity with a “hunched over” appearance. Moribund mice were euthanized based on Emory IACUC end-points and protocols.

Quantitative PCR Amplification of the MCMVDNA:

DNA was isolated from segments of liver using commercially-available reagents (EZNA tissue DNA kit; Omega Bio-Tek, Doraville, Ga.) according to the manufacturer's instructions. DNA was quantitated against a λ DNA standard curve constructed in a microtiter plate-based format (PicoGreen dsDNA Quantitation Kit; Molecular Probes, Eugene, Oreg.). MCMV DNA was quantitated by PCR using a thermocycler interfaced with a fluorescence detection module (iCycler; Bio-Rad Laboratories, Hercules, Calif.) and commercially-available reagents (SYBR Green PCR Master Mix; Applied Biosystems, Foster City, Calif.). In initial validation studies using uninfected liver DNA samples spiked with known amounts of MCMV DNA, the best accuracy and precision were obtained when the samples were assayed in a modified limiting-dilution format. A previously described PCR protocol for amplification of a 363-bp segment of ie1 exon 4 of MCMV using primers IE1.1983 (5′-ATTGTTCATTGCCTGGGGAGTTT-3′) (SEQ ID NO:1) (55) and IE1.2345 (5′-ATCTGGTGCTCCTCAGATCAGCTAA-3′) (SEQ ID NO:2) was modified through empirical trials to increase assay sensitivity and eliminate non-specific amplification for use with SYBR Green detection on the iCycler platform. In the resulting protocol, each 15 μl reaction in a 96-well plate contained a 1:2 dilution of SYBR Green PCR Master Mix, 4.5 pmol IE1.1983, 0.75 pmol IE1.2345, 1 μM MgCl₂, and target DNA. For each liver sample, 10-fold serial dilutions of purified DNA were assayed in quadruplicate starting with 40 ng. Amplification conditions were 95° C.×10 min, followed by 50 cycles of 95° C.×15 sec, 62° C.×15 sec, and 72° C.×60 sec. At the conclusion of PCR, any amplified products were denatured by increasing the temperature stepwise from 60° C. to 99° C. over 39 mins, and the change in fluorescence with increasing temperature was used to generate a melt curve using an Excel macro (SYBRMELT97; Bio-Rad Laboratories). Each 15 μl reaction was then scored as positive or negative based on the presence or absence, respectively, of a characteristic melting temperature for the 363-bp MCMV ie1 amplicon. In initial studies, the accuracy of the melt curve in identifying positive and negative samples was confirmed by the presence or absence of an appropriately sized band on a 5% non-denaturing polyacrylamide gel. This limiting dilution format allowed the number of MCMV genomes in each liver sample to be calculated using the statistical method of Karber (54). This assay can routinely detect 1-5 MCMV genome-equivalents.

Histologic Assessment of GvHD and Viral Cytopathic Effect:

Mice were euthanized, and segments of liver were immersion-fixed in 10% formalin, embedded in paraffin, cut in 8 μm sections, and stained with hematoxylin and eosin. Slides were coded and examined microscopically by one of the co-authors (D.J.) who was blinded to treatment group. Two liver sections per mouse were graded according to criteria previously described (1). Typical eosinophilic intranuclear cytomegalic inclusion bodies were counted in 10 contiguous high-power fields (hpf) on each section as a marker of viral replication. For GvHD, inflammation was graded on a scale of 1-5, with 1=negligible inflammation and 5=dense periportal inflammation spilling into the hepatic lobules. Necrosis was likewise graded on a scale of 1-5, with 1=no necrosis and 5=necrosis of ≧25% of hepatocytes. The results were decoded, tabulated, and analyzed by another co-author (J.R.).

Flow Cytometric Analysis of Leukocyte Chimerism:

These methods have been previously described (1). Briefly, blood was collected at 2-3 months post-BMT and red blood cells (RBCs) were depleted by sequential 1G sedimentation using 3% Dextran T500 in HBSS and subsequent hypotonic saline lysis. Lymphocytes were stained with fluorochrome-conjugated monoclonal antibodies (MoAbs) to distinguish their origin from either BMT recipient, BM donor, or splenocyte donor. MoAbs used included those specific for H2^{b} or H2^{d} MHC, and Thy1.1, Thy1.2, CD45.1, and CD45.2 antigens (Pharmingen). Propidium iodide was added at a concentration of 1 μg/ml, and dead cells were electronically excluded.

Statistical Analyses:

Mantel-Cox log-rank test, Chi-squared test, and Students t-test were applied as described in the text. Significance was demonstrated at the 0.05 level.

Recipients of MHC Mis-Matched BMT have Increased MCMV Lethality.

F1 mice conditioned with y-irradiation underwent BMT from B/6 (allogeneic) or F1 (syngeneic) donors, followed by infection with MCMV inocula ranging from 10⁴ to 5×10⁶ PFU. F1 recipients of syngeneic (FIG. 8A) or allogeneic (FIG. 8B) BMT displayed 100% survival in the absence of MCMV infection (BMT control; closed circle). Following MCMV infection, mortality of B/6→F1 allogeneic BMT recipients was significantly greater than for F1→F1 syngeneic recipients at MCMV inocula of 10⁴ (P<0.001), 5×10⁴ (P<0.001), 10⁵ (P<0.005), and 5×10⁵ PFU (P<0.025) by the Mantel-Cox log-rank test. Infection with 106 or more PFU MCMV was rapidly and uniformly lethal following either allogeneic or syngeneic BMT. Irradiated F1 mice that did not receive BMT (radiation control) had 80-100% mortality at all MCMV inocula tested. Thus, murine BMT accurately models the clinical situation in which recipients of MHC mis-matched allogeneic transplants are more susceptible to lethal CMV disease during the period of immune reconstitution than recipients of syngeneic BMT (56).

Adoptive Immunotherapy with Lymphocytes from MCMV-Immune Donors Prevents MCMV Lethality Without Producing GvHD.

Lymphocytes were prepared from the spleens of naïve or MCMV-immune B/6 donors and 10×10⁶ or 30×10⁶ were infused IV at the time of B/6→F1 allogeneic BMT. Prior to infusion, the splenocytes were either cultured for 24 hours (control) or treated with fludarabine, γ-irradiation, or PCT with S-59 psoralen and ultraviolet A (UVA) light as described in Methods. Some groups of mice were subsequently infected with 2×10⁴ PFU MCMV at two hours after BMT, a viral inoculum expected to produce 90-100% lethality (FIG. 8B).

Three initial experiments using 10×10⁶ B/6 lymphocytes for adoptive immunotherapy produced variable survival results at day +120 post-BMT, depending on the MCMV-immune status of the donor and pretreatment regimen used (Table 2). As compared to MCMV-infected mice that did not receive adoptive immunotherapy (0 of 20 surviving), survival was significantly improved by infusion of immune lymphocytes prior to MCMV infection, regardless of the pretreatment regimen (P<0.001, by chi-squared). While the difference in survival between mice receiving MCMV-immune lymphocytes treated with PCT or gamma-irradiation was significant (P<0.025), the differences between the other groups were not. For all pretreatment regimens, immune lymphocytes were significantly more efficacious at preventing MCMV lethality than were naive cells (Table 2). Among the mice receiving naive cells, significant abrogation of MCMV lethality was only seen with passive transfer of lymphocytes exposed to PCT (P<0.01) or fludarabine (P<0.025). Over the course of these three experiments, all 10 mice irradiated without subsequent BMT died (radiation control) while 19 of 20 mice (95%) survived following irradiation and BMT in the absence of MCMV infection and adoptive immunotherapy (BMT control).

Because 65% of the mice receiving untreated MCMV-immune lymphocytes survived, it was difficult to rigorously assess the ability of different pretreatment regimens to prevent GvHD. In subsequent studies, the lymphocyte dosage was increased to 30×10⁶ cells to increase the probability of GvHD, and only MCMV-immune cells were used since they were significantly more effective than naive lymphocytes in preventing MCMV lethality (Table 2). The results from two separate experiments (n=20 mice per group) are displayed together in FIGS. 2A-C. MCMV infection was lethal in 85% of mice that did not receive adoptive immunotherapy (FIG. 9A, closed squares), while infected mice infused with untreated lymphocytes (FIG. 9A, open triangle) had 100% mortality, due primarily to GvHD (see FIG. 9B). The difference in survival between these groups was not statistically significant (P>0.05). In marked contrast, mice infused with lymphocytes pretreated with fludarabine (open circle), gamma-irradiation (closed triangle), or S-59 psoralen PCT (closed circle) showed significantly improved survival compared to mice that did not receive adoptive immunotherapy (FIG. 9A; P<0.001 by Mantel-Cox log-rank test). Remarkably, 95% of mice infused with MCMV-immune allogeneic PCT lymphocytes survived the lethal MCMV challenge, although survival in this group was not statistically better than groups treated with irradiated or fludarabine-treated lymphocytes (P>0.05). The data in FIG. 9A clearly demonstrated that adoptive immunotherapy with pretreated lymphocytes significantly improved survival of immunocompromised hosts following an MCMV challenge. However, it was unclear whether the residual mortality observed following passive transfer of pretreated lymphocytes was due to viral effects or GvHD. To address this question, similar experiments were performed except without MCMV infection, so that observed lethality could be attributed primarily to GvHD from the passively transferred lymphocytes (FIG. 9B). BMT mice that did not receive adoptive immunotherapy showed 10% mortality (closed squares), while 100% of mice infused with untreated lymphocytes died (open triangles; P<0.001). In contrast, none of the mice receiving lymphocytes pretreated with either S-59 psoralen (closed circle) or γ-irradiation (closed triangle) died (P>0.05, versus mice that did not receive splenocytes). However, 50% of mice infused with fludarabine-pretreated lymphocytes died, a mortality rate significantly greater than with PCT or irradiated lymphocytes (P<0.01). Thus, adoptive immunotherapy with 30×10⁶ MHC mis-matched MCMV-immune lymphocytes pretreated with either S-59 psoralen/UVA light or gamma-irradiation effectively controlled MCMV infection without producing biologically significant GvHD in immunocompromised BMT recipients.

Changes in body weight of mice following BMT is an accepted measure for the development of GvHD. The weight of BMT control mice (FIG. 9C, open squares) decreased transiently during the first week post-BMT, followed by a return to baseline. BMT mice infected with MCMV (closed squares) showed an average 23% weight loss during the period of severe lethality (7-14 days post-BMT), followed by a return to baseline weight among the 3 of 20 mice that survived the infection. Mice infused with untreated lymphocytes following MCMV infection (open triangles) showed 100% mortality, and displayed a marked continued decline in weight throughout 30 days post-BMT period consistent with the development of GvHD. In contrast, mice receiving either PCT (closed circles) or gamma-irradiated lymphocytes (closed triangles) showed weight changes similar to BMT control mice, while adoptive immunotherapy with fludarabine-treated lymphocytes (open circles) showed long-term weight loss suggesting the development of GvHD in this group.

Adoptive Immunotherapy Reduces Hepatic MCMV Viral Load with Limited Hepatic Inflammation.

Because the studies shown in FIG. 9 monitored lethality following adoptive immunotherapy, mice were not sacrificed to provide prospective samples for further analysis. In order to determine the effect of adoptive immunotherapy on MCMV load, B/6→F1 BMT mice, some given adoptive immunotherapy (30×10⁶ lymphocytes) and/or MCMV infection (5×10⁴ PFU), were sacrificed at day +14 and liver samples were analyzed by quantitative MCMV PCR. This time point was chosen because it was prior to the time when the majority of deaths occur post-BMT (FIG. 9A). FIG. 10 shows the combined data from two such experiments (n=10 mice per treatment arm). Only 6 of 10 MCMV-infected BMT mice survived to day +14 in the absence of lymphocyte infusion, and the surviving mice had viral loads averaging 10⁷ copies/40 ng input DNA. In contrast, 80-100% of mice receiving adoptive immunotherapy survived to day +14, and lymphocyte infusion significantly reduced average MCMV load by 4-5 log₁₀ (P<0.05 by Wilcoxon signed ranks test). The difference in viral loads between mice that received PCT lymphocytes or fludarabine-treated lymphocytes was also statistically significant (P<0.005).

A sample of liver was also prepared for microscopic quantitation of MCMV-infected cells and hepatic inflammation. A trained anatomic pathologist (D.J.), blinded to sample identity, scored the sections for MCMV inclusions and hepatic portal inflammation. Representative photomicrographs are shown in FIGS. 11(A-G) and quantitative data analysis in FIG. 11H. Consistent with the viral load data shown in FIG. 10, numerous typical cytomegalic intranuclear inclusions were identified in sections from MCMV-infected mice that did not receive lymphocyte infusions, but not in mice that received adoptive immunotherapy (P<0.005). Evaluation of portal inflammation, as a histologic correlate of GvHD, demonstrated an marked inflammation with an average score of 4.5 (out of 5) in mice receiving untreated lymphocytes. In contrast, mice infused with lymphocytes pretreated with S-59/UVA showed significantly less inflammation (P<0.01) whether MCMV infected (inflammation score of 3.0) or not (2.3). However, the level of inflammation seen following PCT lymphocyte infusion was greater than in BMT mice that did not receive adoptive immunotherapy (P<0.01). Thus, adoptive immunotherapy with pretreated MCMV-immune lymphocytes can markedly reduce MCMV load, which is a primary determinant of clinical CMV disease and mortality in humans (3-6). Furthermore, treatment of the lymphocytes with S-59 psoralen and UVA prior to passive transfer markedly decreases hepatic inflammation and GvHD in the partially MHC mis-matched host.

Pretreatment of Lymphocytes Does Not Prevent Long-Term Persistence Following Adoptive Transfer.

The contribution of the BMT recipient, BMT donor, and splenic lymphocyte donor to the peripheral blood T-cell compartment was determined by flow cytometric immunophenotyping at days 60 and 90 post-BMT (FIG. 12). In all groups tested, at least 50% of circulating T-cells were derived from the BM donor, and the remaining T-cells were variably accounted for by either the adoptively transferred T-cells lymphocytes or residual lymphocytes from the BMT recipient. Interestingly, mice that received MCMV-immune lymphocytes pretreated with either psoralen or gamma-irradiation demonstrated greater persistence of adoptively-transferred T-cells, and fewer T-cells derived from the BMT recipient, in the presence of concurrent MCMV infection. Mice infused with fludarabine pretreated lymphocytes had an equivalent percentage of T-cells derived from the BM and lymphocyte donor, in the absence of detectable recipient T-cells. Thus, pretreated mature T-cells can persist long-term in MHC mismatched recipients following adoptive transfer, and their survival may be correlated with their role in an immune response.

Throughout this application, various publications, patents, and/or patent applications are referenced in order to more fully describe the state of the art to which this invention pertains. The disclosures of these publications, patents, and/or patent applications are herein incorporated by reference in their entireties to the same extent as if each independent publication, patent, and/or patent application was specifically and individually indicated to be incorporated by reference.

### REFERENCES

1. Waller E K, Ship A M, Mittelstaedt S, et al. Irradiated donor leukocytes promote engraftment of allogeneic bone marrow in major histocompatibility complex mismatched recipients without causing graft-versus-host disease. Blood 94:3222-33, 1999.

2. Waller E K, Boyer M. New strategies in allogeneic stem cell transplantation: immunotherapy using irradiated allogeneic T cells. Bone Marrow Transplant Suppl 2:S20-4, 2000.

3. Roux E, Abdi K, Speiser D, et al. Characterization of mixed chimerism in patients with chronic myeloid leukemia transplanted with T-cell-depleted bone marrow: involvement of different hematologic lineages before and after relapse. Blood 81:243-8, 1993.

4. Avramis V I, Plunkett W: 2-fluoro-ATP. a toxic metabolite of 9-beta-D-arabinosyl-2-fluoroadenine. Biochemical & Biophysical Research Communications 113:35-43, 1983.

5. Huang P, Chubb S, Plunkett W. Termination of DNA synthesis by 9-beta-D-arabinofuranosyl-2-fluoroadenine. A mechanism for cytotoxicity. J Biol Chem 265:16617-25, 1990.

6. Priebe T, Platsoucas C D, Seki H, Fox F E, Nelson J A. Purine nucleoside modulation of functions of human lymphocytes. Cellular Immunology 129:321-8., 1990.

7. Cheson BD: Infectious and immunosuppressive complications of purine analog therapy. Journal of Clinical Oncology 13:2431-48, 1995.

8. Sandoval A, Consoli U, Plunkett W. Fludarabine-mediated inhibition of nucleotide excision repair induces apoptosis in quiescent human lymphocytes. Clinical Cancer Research 2:1731-41, 1996.

9. Frank DA, Mahajan S, Ritz J. Fludarabine-induced immunosuppression is associated with inhibition of STATI signaling. Nature Medicine. 5:444-7, 1999.

10. Truitt R, Johnson B, Hanke C, Talib S, Hearst J. Photochemical treatment with S-59 psoralen and ultraviolet A light to control the fate of naive or primed T lymphocytes in vivo after allogeneic bone marrow transplantation. Journal of Immunology 163:5145-56, 1999.

11. Turner C W, Yeager A M, Waller E K, Wingard J R, Fleming W H. Engraftment potential of different sources of human hematopoietic progenitor cells in BNX Mice. Blood 87:3237-44, 1996.

12. Taswell C. Limiting dilution assays for the determination of immunocompetent cell frequencies. I. Data analysis. J Immunol 126:1614-9, 1981.

13. Wunderlich J, Shearer G. Induction and measurement of cytotoxic T lymphocyte activity: Current Protocols in Immunology, vol. 1, Green Publishing and Wiley-Interscience, p 3.11.1, 1991.

14. Cox D R, Oakes D. Proportional Hazards Model: Analysis os Survival Data. New York, Chapman and Hall, p 104-106, 1984.

15. Gillis S, Mizel S B. T-Cell lymphoma model for the analysis of interleukin 1-mediated T-cell activation. PNAS 78:1133-7, 1981.

16. Johnson B D, Truitt R L. Delayed infusion of immunocompetent donor cells after bone marrow transplantation breaks graft-host tolerance allows for persistent antileukemic reactivity without severe graft-versus-host disease. Blood 85:3302-3312, 1995.

17. Barrett A J, Mavroudis D, Tisdale J, et al. T cell-depleted bone marrow transplantation and delayed T cell add-back to control acute GVHD and conserve a graft-versus-leukemia effect. Bone Marrow Transplantation 21:543-51, 1998.

18. Kolb H, Schattenberg A, Goldman J, et al. Graft-versus-leukemia effect of donor lymphocyte transfusions in marrow grafted patients. Blood. 86:2041-2050, 1995.

19. Collins R H, Jr., Shpilberg O, Drobyski W R, et al. Donor leukocyte infusions in 140 patients with relapsed malignancy after allogeneic bone marrow transplantation. Journal of Clinical Oncology 15:433-44, 1997.

20. Kolb H J, Mittermuller J, Clemm C, et al. Donor leukocyte transfusions for treatment of recurrent chronic myelogenous leukemia in marrow transplant patients. Blood 76:2462-5, 1990.

21. Blazar B R, Lees C J, Martin P J, et al. Host T cells resist graft-versus-host disease mediated by donor leukocyte infusions. J Immunol 165:4901-9, 2000.

22. Koh M B, Prentice H G, Lowdell M W. Selective removal of alloreactive cells from haematopoietic stem cell grafts: graft engineering for GVHD prophylaxis. Bone Marrow Transplantation 23:1071-9, 1999.

23. van Dijk A M, Kessler F L, Stadhouders-Keet S A, Verdonck L F, de Gast G C, Otten H G: Selective depletion of major and minor histocompatibility antigen reactive T cells: towards prevention of acute graft-versus-host disease. British Journal of Haematology 107:169-75, 1999.

24. Bordignon C, Bonini C, Verzeletti S, et al. Transfer of the HSV-tk gene into donor peripheral blood lymphocytes for in vivo modulation of donor anti-tumor immunity after allogeneic bone marrow transplantation. Human Gene Therapy 6:813-819, 1995.

25. Bonini C, Bordignon C: Potential and limitations of HSV-TK-transduced donor peripheral blood lymphocytes after allo-BMT. Hematology & Cell Therapy 39:273-4, 1997.

26. Bonini C, Ferrari G, Verzeletti S, et al. HSV-TK gene transfer into donor lymphocytes for control of allogeneic graft-versus-leukemia. Science 276:1719-24, 1997.

27. Bachar-Lustig E, Li H W, Gur H, Krauthgamer R, Marcus H, Reisner Y. Induction of donor-type chimerism and transplantation tolerance across major histocompatibility barriers in sublethally irradiated mice by Sca-1(+)Lin(−) bone marrow progenitor cells: synergism with non-alloreactive (host x donor)F(1) T cells. Blood 94:3212-21, 1999.

28. Drobyski W R, Majewski D, Ozker K, Hanson G. ex vivo anti-CD3 antibody-activated donor T cells have a reduced ability to cause lethal murine graft-versus-host disease but retain their ability to facilitate alloengraftment. Journal of Immunology 161:2610-9, 1998.

29. Ship A M, Waller E K. Clinical use of irradiated donor lymphocytes in bone marrow transplantation. Cancer Treat Res 101:267-82, 1999.

30. Grass J A, Wafa T, Reames A, et al. Prevention of transfusion-associated graft-versus-host disease by photochemical treatment. Blood 93:3140-7, 1999.

31. Huang P, Plunkett W: Fludarabine- and gemcitabine-induced apoptosis: incorporation of analogs into DNA is a critical event. Cancer Chemotherapy and Pharmacology 36:181-8, 1995.

32. Meyers, J. D., N. Flournoy, and E. D. Thomas. 1986. Risk factors for cytomegalovirus infection after human marrow transplantation. J Infect Dis. 153:478-88.

33. Grundy, J. E., S. F. Lui, M. Super, N. J. Berry, P. Sweny, O. N. Fernando, J. Moorhead, and P. D. Griffiths. 1988. Symptomatic cytomegalovirus infection in seropositive kidney recipients: reinfection with donor virus rather than reactivation of recipient virus. Lancet. 2:132-5.

34. Spector, S. A., R. Wong, K. Hsia, M. Pilcher, and M. J. Stempien. 1998. Plasma cytomegalovirus (CMV) DNA load predicts CMV disease and survival in AIDS patients. J Clin Invest. 101:497-502.

35. Gor, D., C. Sabin, H. G. Prentice, N. Vyas, S. Man, P. D. Griffiths, and V. C. Emery. 1998. Longitudinal fluctuations in cytomegalovirus load in bone marrow transplant patients: relationship between peak virus load, donor/recipient serostatus, acute GVHD and CMV disease. Bone Marrow Transplant. 21:597-605.

36. Hassan-Walker, A. F., I. M. Kidd, C. Sabin, P. Sweny, P. D. Griffiths, and V. C. Emery. 1999. Quantity of human cytomegalovirus (CMV) DNAemia as a risk factor for CMV disease in renal allograft recipients: relationship with donor/recipient CMV serostatus, receipt of augmented methylprednisolone and antithymocyte globulin (ATG). J Med Virol. 58:182-7.

37. Emery, V. C., C.A. Sabin, A. V. Cope, D. Gor, A. F. Hassan-Walker, and P. D. Griffiths. 2000. Application of viral-load kinetics to identify patients who develop cytomegalovirus disease after transplantation. Lancet. 355:2032-6.

38. Drew, W. L., R. C. Miner, D. F. Busch, S. E. Follansbee, J. Gullett, S. G. Mehalko, S. M. Gordon, W. F. Owen, T. R. Matthews, W. C. Buhles, and et al. 1991. Prevalence of resistance in patients receiving ganciclovir for serious cytomegalovirus infection. J Infect Dis. 163:716-9.

39. Eckle, T., L. Prix, G. Jahn, T. Klingebiel, R. Handgretinger, B. Selle, and K. Hamprecht. 2000. Drug-resistant human cytomegalovirus infection in children after allogeneic stem cell transplantation may have different clinical outcomes. Blood. 96:3286-9.

40. Goodrich, J. M., R. A. Bowden, L. Fisher, C. Keller, G. Schoch, and J. D. Meyers. 1993. Ganciclovir prophylaxis to prevent cytomegalovirus disease after allogeneic marrow transplant. Ann Intern Med. 118:173-8.

41. Winston, D. J., W. G. Ho, K. Bartoni, C. Du Mond, D. F. Ebeling, W. C. Buhles, and R. E. Champlin. 1993. Ganciclovir prophylaxis of cytomegalovirus infection and disease in allogeneic bone marrow transplant recipients. Results of a placebo-controlled, double-blind trial. Ann Intern Med. 118:179-84.

42. Prentice, H. G., and P. Kho. 1997. Clinical strategies for the management of cytomegalovirus infection and disease in allogeneic bone marrow transplant. Bone Marrow Transplant. 19:135-42.

43. Jonjic, S., W. Mutter, F. Weiland, M. J. Reddehase, and U. H. Koszinowski. 1989. Site-restricted persistent cytomegalovirus infection after selective long-term depletion of CD4+T lymphocytes. J Exp Med. 169:1199-212.

44. Steffens, H. P., S. Kurz, R. Holtappels, and M. J. Reddehase. 1998. Preemptive CD8 T-cell immunotherapy of acute cytomegalovirus infection prevents lethal disease, limits the burden of latent viral genomes, and reduces the risk of virus recurrence. J Virol. 72:1797-804.

45. Quinnan, G. V., N. Kirmani, A. H. Rook, J. F. Manischewitz, L. Jackson, G. Moreschi, G. W. Santos, R. Saral, and W. H. Bums. 1982. Cytotoxic T cells in cytomegalovirus infection: HLA-restricted T-lymphocyte and non-T-lymphocyte cytotoxic responses correlate with recovery from cytomegalovirus infection in bone-marrow-transplant recipients. N Engl J Med. 307:7-13.

46. Quinnan, G. V., W. H. Bums, N. Kirmani, A. H. Rook, J. Manischewitz, L. Jackson, G. W. Santos, and R. Saral. 1984. HLA-restricted cytotoxic T lymphocytes are an early immune response and important defense mechanism in cytomegalovirus infections. Rev Infect Dis. 6:156-63.

47. Reusser, P., S. R. Riddell, J. D. Meyers, and P. D. Greenberg. 1991. Cytotoxic T-lymphocyte response to cytomegalovirus after human allogeneic bone marrow transplantation: pattern of recovery and correlation with cytomegalovirus infection and disease. Blood. 78:1373-80.

48. Li, C. R., P. D. Greenberg, M. J. Gilbert, J. M. Goodrich, and S. R. Riddell. 1994. Recovery of HLA-restricted cytomegalovirus (CMV)-specific T-cell responses after allogeneic bone marrow transplant: correlation with CMV disease and effect of ganciclovir prophylaxis. Blood. 83:1971-9.

49. Riddell, S. R., and P. D. Greenberg. 1995. Principles for adoptive T cell therapy of human viral diseases. Annu Rev Immunol. 13:545-86.

50. Storb, R., K. C. Doney, E. D. Thomas, F. Appelbaum, C. D. Buckner, R. A. Clift, H. J. Deeg, B. W. Goodell, R. Hackman, J. A. Hansen, J. Sanders, K. Sullivan, P. L. Weiden, and R. P. Witherspoon. 1982. Marrow transplantation with or without donor buffy coat cells for 65 transfused aplastic anemia patients. Blood. 59:236-46.

51. Riddell, S. R., K. S. Watanabe, J. M. Goodrich, C. R. Li, M. E. Agha, and P. D. Greenberg. 1992. Restoration of viral immunity in immunodeficient humans by the adoptive transfer of T cell clones [see comments]. Science. 257:238-41.

52. Walter, E. A., P. D. Greenberg, M. J. Gilbert, R. J. Finch, K. S. Watanabe, E. D. Thomas, and S. R. Riddell. 1995. Reconstitution of cellular immunity against cytomegalovirus in recipients of allogeneic bone marrow by transfer of T-cell clones from the donor [see comments]. N Engl J Med. 333:1038-44.

53. Kurz, S., H. P. Steffens, A. Mayer, J. R. Harris, and M. J. Reddehase. 1997. Latency versus persistence or intermittent recurrences: evidence for a latent state of murine cytomegalovirus in the lungs. J Virol. 71:2980-7.

54. Payment, P., and M. Trudel. 1993. Isolation and identification of viruses. In Methods and techniques in virology. P. Payment, and M. Trudel, editors. Marcel Dekker, New York. 33.

55. Balthesen, M., M. Messerle, and M. J. Reddehase. 1993. Lungs are a major organ site of cytomegalovirus latency and recurrence. J Virol. 67:5360-6.

56. Wingard, J. R., D. Y. Chen, W. H. Burns, D. J. Fuller, H. G. Braine, A. M. Yeager, H. Kaiser, P. J. Burke, M. L. Graham, G. W. Santos, and et al. 1988. Cytomegalovirus infection after autologous bone marrow transplantation with comparison to infection after allogeneic bone marrow transplantation. Blood. 71: 1432-7.

57. Chan, K. W., C. J. Fryer, J. F. Denegri, N. A. Buskard, and G. L. Phillips. 1987. Allogeneic bone marrow transplantation using partially-matched related donors. Bone Marrow Transplant. 2:27-32.

58. Anasetti, C., D. Amos, P. G. Beatty, F. R. Appelbaum, W. Bensinger, C. D. Buckner, R. Clift, K. Doney, P. J. Martin, E. Mickelson, and et al. 1989. Effect of HLA compatibility on engraftment of bone marrow transplants in patients with leukemia or lymphoma. N Engl J Med. 320:197-204.

59. Bosserman, L. D., C. Murray, T. Takvorian, K. C. Anderson, A. S. Freedman, J. Fitzsimmons, F. Coral, L. M. Nadler, S. F. Schlossman, and J. Ritz. 1989. Mechanism of graft failure in HLA-matched and HLA-mismatched bone marrow transplant recipients. Bone Marrow Transplant. 4:239-45.

60. Henslee-Downey, P. J., S. H. Abhyankar, R. S. Parrish, A. R. Pati, K. T. Godder, W. J. Neglia, K. S. Goon-Johnson, S. S. Geier, C. G. Lee, and A. P. Gee. 1997. Use of partially mismatched related donors extends access to allogeneic marrow transplant. Blood. 89:3864-72.

61. Lienert-Weidenbach, K., N. M. Valiante, C. Brown, C. White, L. Johnston-Dow, M. McGinnis, P. Krausa, D. M. Lakes, J. L. Wolf, K. G. Blume, and P. Parham. 1997. Mismatches for two major and one minor histocompatibility antigen correlate with a patient's rejection of a bone marrow graft from a serologically HLA-identical sibling. Biol Blood Marrow Transplant. 3:255-60.

62. Aversa, F., A. Tabilio, A. Terenzi, A. Velardi, F. Falzetti, C. Giannoni, R. Iacucci, T. Zei, M. P. Martelli, C. Gambelunghe, and et al. 1994. Successful engraftment of T-cell-depleted haploidentical “three-loci” incompatible transplants in leukemia patients by addition of recombinant human granulocyte colony-stimulating factor-mobilized peripheral blood progenitor cells to bone marrow inoculum. Blood. 84:3948-55.

63. Keever, C. A., T. N. Small, N. Flomenberg, G. Heller, K. Pekle, P. Black, A. Pecora, A. Gillio, N. A. Kernan, and R. J. O'Reilly. 1989. Immune reconstitution following bone marrow transplantation: comparison of recipients of T-cell depleted marrow with recipients of conventional marrow grafts. Blood. 73:1340-50.

64. Aversa, F., A. Tabilio, A. Velardi, I. Cunningham, A. Terenzi, F. Falzetti, L. Ruggeri, G. Barbabietola, C. Aristei, P. Latini, Y. Reisner, and M. F. Martelli. 1998. Treatment of high-risk acute leukemia with T-cell-depleted stem cells from related donors with one fully mismatched HLA haplotype [see comments]. N Engl J Med. 339:1186-93.

TABLE 1
Splenocyte viability in 24 hour culture (% compared to starting cell no.)

| Cell population | 0 μg/ml Fludarabine | 20 μg/ml Fludarabine |
|---|---|---|
| All leukocytes | 74.5 ± 2.9% | 41.6 ± 7.8% |
| CD4 + T-cells | 84.3 ± 4.6% | 69.0 ± 9.9% |
| CD8 + T-cells | 81.2 ± 3.7% | 62.1 ± 12.9% |
| Non T-cells | 71.3 ± 4.8% | 33.6 ± 6.4% |

Table 1. Cell viability after 24 hr culture with or without 20 μg/ml fludarabine. Results show the percentages of T-cell and non-T-cell populations remaining viable after the culture period. The data represent the mean ±SEM for 4 individual experiments.

TABLE 2

| | | | | | Immune vs. |
|---|---|---|---|---|---|
| | Immune splenocytes | | Naive splenocytes | | Naive |
| Treatment | Survivors/Total | % | Survivors/Total | % | P value |
| S-59 | 9/10 | 90 | 3/10 | 30 | <0.01 |
| Fludarabine | 24/34 | 71 | 8/35 | 23 | <0.001 |
| Irradiated | 9/19 | 47 | 1/20 | 5 | <0.005 |
| Untreated | 13/20 | 65 | 2/20 | 10 | <0.001 |

Table 2: Effect of adoptive immunotherapy on the survival of B/6→F1 BMT mice following MCMV infection. At the time of BMT, mice were infused with 10×10⁶ splenic lymphocytes from B/6 mice that were either previously-infected with MCMV (“Immune splenocytes”) or uninfected (“Naive splenocytes”). Prior to infusion, the lymphocytes were either treated with S-59/UVA, gamma-irradation, or fludarabine. Some mice received lymphocytes that were untreated. The table lists the number of surviving mice (at day +120 post-BMT) and the total number of mice in each group, as well as the percentage survival. As compared to MCMV-infected BMT mice that did not receive adoptive immunotherapy (0 of 20 surviving), survival was significantly improved by infusion by 10×10⁶ immune lymphocytes prior to MCMV infection, regardless of the pretreatment regimen (P<0.001, by chi-squared). For each treatment group, survival was significantly better following infusion of immune splenocytes than with naive splenocytes (P values listed in table).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of transplanting hematopoietic cells from a donor source into a genetically unrelated recipient, comprising:
a) administering to the recipient, in combination with the administration of the hematopoietic cells, an amount of mononuclear cells which are treated so as to substantially reduce their ability to cause graft versus host disease while they retain their ability to proliferate in the recipient and facilitate engraftment of the hematopoietic cells in the recipient; and
b) administering to the recipient an effective amount of hematopoietic cells.

2. The method of claim 1, wherein the mononuclear cells are r cells.

3. The method of claim 1, wherein the mononuclear cells are natural killer cells.

4. The method of claim 1, wherein the mononuclear cells are a mixture of T cells and natural killer cells.

5. The method of claim 1, wherein the cells are treated with a chemotherapeutic agent.

6. The method of claim 5, wherein the chemotherapeutic agent is selected from the group consisting of 9-D-arabinofuranosyl-2-fluoroadenosinemonophosphate (fludarabine), 2′-deoxcoformycin (pentostatin), 2-chlorodeoxyadenosine (2CDA), 6-mercaptopurine (6-MP), 6-thioguanine (6-TG), 2′-deoxy-2′, 2′-difluorocytidine (gemcitabine) and 2-amino-9-D-arabinosyl-6-methoxy-9-H-purine (Ara-G, 506U78).

7. A method of treating or preventing an infection in a recipient of genetically unrelated hematopoietic cells, comprising administering to the recipient, in combination with the administration of the hematopoietic cells, an amount of mononuclear cells which are treated so as to substantially reduce their ability to cause graft versus host disease while they retain their ability to proliferate in the recipient, and which are effective in treating or preventing the infection.

8. The method of claim 7, wherein the mononuclear cells are T cells.

9. The method of claim 7, wherein the mononuclear cells are natural killer cells.

10. The method of claim 7, wherein the mononuclear cells are a mixture of T cells and natural killer cells.

11. The method of claim 7, wherein the cells are treated with a chemotherapeutic agent.

12. The method of claim 11, wherein the chemotherapeutic agent is selected from the group consisting of 9-D-arabinofuranosyl-2-fluoroadenosinemonophosphate (fludarabine), 2′-deoxcoformycin (pentostatin), 2-chlorodeoxyadenosine (2CDA), 6-mercaptopurine (6-MP), 6-thioguanine (6-TG), 2′-deoxy-2′,2′-difluorocytidine (gemcitabine) and 2-amino-9-D-arabinosyl-6-methoxy-9-H-purine (Ara-G, 506U78).

13. The method of claim 7, wherein the infection is caused by a virus.

14. The method of claim 13, wherein the virus is cytomegalovirus.

15. A method of enhancing immune reconstitution in a transplant recipient, comprising administering to the recipient, in combination with a transplant, an amount of mononuclear cells which are treated so as to substantially reduce their ability to cause graft versus host disease while they retain their ability to proliferate in the recipient, and which are effective in enhancing immune reconstitution in the recipient.

16. The method of claim 15, wherein the mononuclear cells are T cells.

17. The method of claim 15, wherein the mononuclear cells are natural killer cells.

18. The method of claim 15, wherein the mononuclear cells are a mixture of T cells and natural killer cells.

19. The method of claim 15, wherein the cells are treated with a chemotherapeutic agent.

20. The method of claim 19, wherein the chemotherapeutic agent is selected from the group consisting of 9-D-arabinofuranosyl-2-fluoroadenosinemonophosphate (fludarabine), 2′-deoxcoformycin (pentostatin), 2-chlorodeoxyadenosine (2CDA), 6-mercaptopurine (6-MP), 6-thioguanine (6-TG), 2′-deoxy-2′,2′-difluorocytidine (gemcitabine) and 2-amino-9-D-arabinosyl-6-methoxy-9-H-purine (Ara-G, 506U78).

21. A method of enhancing immune reconstitution in a subject diagnosed with cancer, comprising administering to the subject an amount of mononuclear cells which are treated so as to substantially reduce their ability to cause graft versus host disease while they retain their ability to proliferate in the subject, and which are effective in enhancing immune reconstitution in the subject.

22. The method of claim 21, wherein the mononuclear cells are T cells.

23. The method of claim 21, wherein the mononuclear cells are natural killer cells.

24. The method of claim 21, wherein the mononuclear cells are a mixture of T cells and natural killer cells.

25. The method of claim 21, wherein the cancer originates in a solid organ.

26. The method of claim 21, wherein the cancer originates in hematopoietic tissue.

27. The method of claim 21, wherein the cancer is metastatic.

28. The method of claim 21, wherein the cells are treated with a chemotherapeutic agent.

29. The method of claim 28, wherein the chemotherapeutic agent is selected from the group consisting of 9-D-arabinofuranosyl-2-fluoroadenosinemonophosphate (fludarabine), 2′-deoxcoformycin (pentostatin), 2-chlorodeoxyadenosine (2CDA), 6-mercaptopurine (6-MP), 6-thioguanine (6-TG), 2′-deoxy-2′,2′-difluorocytidine (gemcitabine), 2-amino-9-D-arabinosyl-6-methoxy-9-H-purine (Ara-G, 506U78) and S-59 psoralen activated by ultraviolet A light.

30. A method of treating or preventing an infection in a genetically unrelated solid organ transplant recipient, comprising administering to the recipient, in combination with the transplant, an amount of mononuclear cells which are treated so as to substantially reduce their ability to cause graft versus host disease while they retain their ability to proliferate in the recipient, and which are effective in treating or preventing the infection.

31. The method of claim 30, wherein the mononuclear cells are T cells.

32. The method of claim 30, wherein the mononuclear cells are natural killer cells.

33. The method of claim 30, wherein the mononuclear cells are a mixture of T cells and natural killer cells.

34. The method of claim 30, wherein the infection is caused by a virus.

35. The method of claim 34, wherein the virus is cytomegalovirus.

36. The method of claim 30, wherein the cells are treated with a chemotherapeutic agent.

37. The method of claim 36, wherein the chemotherapeutic agent is selected from the group consisting of 9-D-arabinofuranosyl-2-fluoroadenosinemonophosphate (fludarabine), 2′-deoxcoformycin (pentostatin), 2-chlorodeoxyadenosine (2CDA), 6-mercaptopurine (6-MP), 6-thioguanine (6-TG), 2′-deoxy-2′,2′-difluorocytidine (gemcitabine) and 2-amino-9-D-arabinosyl-6-methoxy-9-H-purine (Ara-G, 506U78) and psoralen activated by ultraviolet A light.

38. A method of treating or preventing an infection in a subject, comprising administering to the subject an amount of mononuclear cells which are treated so as to substantially reduce their ability to cause graft versus host disease while they retain their ability to proliferate in the subject, and which are effective in treating or preventing the infection.

39. The method of claim 38, wherein the mononuclear cells are T cells.

40. The method of claim 38, wherein the mononuclear cells are natural killer cells.

41. The method of claim 38, wherein the mononuclear cells are a mixture of T cells and natural killer cells.

42. The method of claim 38, wherein the subject is immunocompetent.

43. The method of claim 42, wherein the subject is HIV positive.

44. The method of claim 38, wherein the subject is immunocompromised.

45. The method of claim 44, wherein the subject is HIV positive.

46. The method of claim 38, wherein the subject is a neonate.

47. The method of claim 38, wherein the subject requires augmentation of cellular immunity.

48. The method of claim 38, wherein the infection is caused by a virus.

49. The method of claim 48, wherein the virus is cytomegalovirus.

50. The method of claim 38, wherein the cells are treated with a chemotherapeutic agent.

51. The method of claim 50, wherein the chemotherapeutic agent is selected from the group consisting of 9-D-arabinofuranosyl-2-fluoroadenosinemonophosphate (fludarabine), 2′-deoxcoformycin (pentostatin), 2-chlorodeoxyadenosine (2CDA), 6-mercaptopurine (6-MP), 6-thioguanine (6-TG), 2′-deoxy-2′,2′-difluorocytidine (gemcitabine), 2-amino-9-D-arabinosyl-6-methoxy-9-H-purine (Ara-G, 506U78) and S-59 psoralen activated by ultraviolet A light.

52. A method of treating cancer in a subject diagnosed with a cancer, comprising administering to the subject an amount of mononuclear cells which are treated so as to substantially reduce their ability to cause graft versus host disease while they retain their ability to proliferate in the subject, and which are effective in treating the cancer.

53. The method of claim 52, wherein the mononuclear cells are T cells.

54. The method of claim 52, wherein the mononuclear cells are natural killer cells.

55. The method of claim 52, wherein the mononuclear cells are a mixture of T cells and natural killer cells.

56. The method of claim 52, wherein the cancer is leukemia.

57. The method of claim 52, wherein the cells are treated with a chemotherapeutic agent.

58. The method of claim 57, wherein the chemotherapeutic agent is selected from the group consisting of 9-D-arabinofuranosyl-2-fluoroadenosinemonophosphate (fludarabine), 2′-deoxcoformycin (pentostatin), 2-chlorodeoxyadenosine (2CDA), 6-mercaptopurine (6-MP), 6-thioguanine (6-TG), 2′-deoxy-2′,2′-difluorocytidine (gemcitabine), 2-amino-9-D-arabinosyl-6-methoxy-9-H-purine (Ara-G, 506U78) and S-59 psoralen activated by ultraviolet A light.
